(19) United States
(12) Patent Application Publication
(10) Pub. No.: US 2025/0073194 A1
(43) Pub. Date: Mar. 6, 2025

(54) RXR AGONISTS IN EYE DISORDERS

(71) Applicant: Baylor College of Medicine, Houston, TX (US)

(72) Inventors: Stephen C. Pflugfelder, Houston, TX (US); Jahan Alam, Houston, TX (US)

(73) Assignee: Baylor College of Medicine, Houston, TX (US)

(21) Appl. No.: 18/726,702

(22) PCT Filed: Feb. 1, 2023
(86) PCT No.: PCT/US2023/061763

§ 371 Date: Jul. 3, 2024

Related U.S. Application Data
(60) Provisional application No. 63/305,596, filed on Feb. 1, 2022.

(51) Int. Cl. A61K 31/203; A61K 31/05; A61K 31/192; A61K 31/201; A61K 31/202; A61K 45/06

(52) U.S. Cl. CPC A61K 31/203; A61K 31/05; A61K 31/192; A61K 31/201; A61K 31/202; A61K 45/06

(57) ABSTRACT

Embodiments of the present disclosure concern the treatment and/or prevention of eye disorders, including dry eye disorders and any medical condition that has dry eye as a symptom. In specific embodiments, treatment and/or prevention may occur by administering therapeutic compositions comprising one or more RXR agonists to at least one eye of an individual.

This application claims priority to U.S. Provisional Patent Application Ser. No. 63/305,596, filed Feb. 1, 2022, which is incorporated by reference herein in its entirety.

This invention was made with government support under EY011915 awarded by the National Institutes of Health. The government has certain rights in the invention.

### BACKGROUND

### I. Technical Field

This disclosure relates at least to the field of cell biology, biochemistry, molecular biology, medicine, and ophthalmology.

### II. Background

Dry eye disorders are prevalent, affecting tens of millions of individuals worldwide. (Pflugfelder and de Paiva, 2017) Clinical trial results and animal models provide evidence that inflammation contributes to the pathogenesis of ocular surface disease in dry eye. (Perez et al., 2020) The ocular surface is an exposed mucosal tissue that is subjected to desiccating and osmotic stress, as well as microbial danger signals. The conjunctiva has a complement of immune cells that produce factors capable of suppressing sight-threatening inflammation during homeostasis but can respond to pathogen and environmental danger signals. Indeed, ocular surface desiccation has been found to be a potent inflammatory stress that stimulates activation of and production of inflammatory mediators (cytokines, chemokines and proteases) by the ocular surface epithelial and inflammatory cells. (Pflugfelder and de Paiva, 2017) This can cause clinical signs of dry eye, such as corneal barrier disruption and conjunctival goblet cell loss. (Alam et al., 2020a; Pflugfelder and Stern, 2020)

The Lacrimal Functional Unit regulates production and distribution of tears containing factors that maintain ocular surface epithelial health and suppress ocular surface inflammation. (Stern et al., 1998) One such lacrimal gland secreted factor is vitamin A in the form of retinol that is metabolized to retinoic acid (RA) by the ocular surface epithelium, particularly the conjunctival goblet cells which can deliver it to immune cells located in the underlying stroma. (Xiao et al., 2018; Pflugfelder and de Paiva, 2020) Dry eye with corneal and conjunctival epithelial disease develops in systemic vitamin A deficiency. However, the pathogeneic mechanisms have not been elucidated. Vitamin A signals through two families of nuclear receptors, the retinoid acid receptor (RAR) and the retinoid X receptor (RXR) that consist as homo- or heterodimers (partners include, RAR, PPAR, vitamin D receptor, and others). (Alam et al., 2021b) RXRα is expressed by a variety of immune cells, including myeloid and lymphoid lineages (Fritsche et al., 2000; Roszer et al., 2013; Raverdeau and Mills, 2014) and myeloid cells in the conjunctiva. (Alam et al., 2021b) Mice with loss of function mutation in the RXRα nuclear receptor have been reported to develop dry eye. (Du et al., 2005)

The disclosure herein describes mechanisms for dry eye development with RXRα loss of function, and compositions and methods for alleviating, preventing, and/or treating such mechanisms. One such mechanism includes the increased population of IL-17-producing γδ T cells (γδ T17 cells) in the dry eye environment with reduced RXRα signaling that promotes development of dry eye disease. The present disclosure satisfies a long-felt need in the art of dry eye treatment.

### BRIEF SUMMARY

Embodiments of the present disclosure concern compositions, and methods utilizing such compositions, comprising at least one RXR agonist. In some embodiments, the composition, which may be a therapeutic composition, is administered to an individual to treat or prevent an eye disorder. In some embodiments, the eye disorder is dry eye disease, Sjogren Syndrome, Meibomian gland disease, tear instability, unstable tear film, tear dysfunction, or one or more ocular surface inflammatory conditions. The individual may have or may be at risk for having dry eye disease, Sjogren's Syndrome, Meibomian gland disease, unstable tear film, tear dysfunction, or an ocular surface inflammatory condition, vitamin A deficiency, chemical corneal injury, thermal corneal injury, cornea inflammation following bacterial, fungal or viral infection, corneal neovascularization, or a combination thereof.

In some embodiments, the RXR agonist comprises an RXRα agonist of any kind. The RXR agonist may comprise 9-cis retinoic acid, oleic acid, omega-3 docosahexaenoic acid, vitamin D, bexarotene, taxerotene, honokiol, AM80, rosiglitazone, Drupanin, garcinoic acid, 4-(ethyl(3-isobutoxy-4-isopropylphenyl)amino) benzoic acid (NEt-3IB), or a combination thereof.

One or both eyes may be affected with an eye disorder or as a result of having a medical condition that does not primarily target the eyes but the eyes are secondarily affected. The composition may be administered to one or both eyes of an individual. In some embodiments, the composition is administered as an eye drop, which may or may not comprise a microdrop. In some embodiments, the composition is administered as a suspension, nanoparticle, ointment, cream, or a combination thereof. In some embodiments, the composition is administered by subconjunctival injection.

The composition may be administered at any dose capable of treating or preventing an eye disorder. In some embodiments, the composition comprises approximately 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600,700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 ng or μg of one or more RXR agonists. In some embodiments, the composition comprises approximately 0.01 ng/mL-50 ng/mL, or any range derivable therein such as 0.03 ng/mL-30 ng/mL, of one or more RXR agonists. In some embodiments, such as for topical administration of an RXR agonist, the concentration of the RXR agonist is approximately 0.03 ng/mL-30 ng/mL in the topical formulation.

In some embodiments, the individual has a condition that predisposes the individual to the eye disorder or to having dry eye(s). The condition may comprise Sjogren syndrome, rheumatoid arthritis, systemic lupus, erythematosus, systemic sclerosis, graft versus host disease, and/or Stevens-Johnson syndrome.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1D show the Pinkie strain with reduced Rxrα signaling develops dry eye disease. 1A) Dry eye phenotype in Pinkie strain. Representative Oregon Green dextran (OGD) staining of corneas from 8- and 32-week-old C57BL/6 (B6) and Pinkie strains (left) and graph showing mean gray level fluorescence (n=9-22/group). Mean±SD, ***P<0.01;****P<0.0001; 1B) Representative images of PAS staining of conjunctival goblet cells in paraffin sections prepared from B6 and Pinkie strains (left) with bar graph of mean goblet cell density (right), n=5/group; 1C) Representative SPPR2 immunostaining of whole mount conjunctivas stained with SPRR2 polyclonal antibody that recognizes multiple isoforms and nuclei stained with Hoechst 33343 dye. Images are captured by confocal microscopy; relative fold expression of Sprr2g, Sprr2f and Sprr2a genes in Pinkie conjunctiva, n=5/group; 1D) Flow cytometry scatter plot showing increased percentage of CD45⁺ cells in the Pinkie conjunctiva (left) and bar graph comparing CD45⁺ cells in conjunctiva obtained from B6 and Pinkie (n=7);

FIGS. 2A-2E show single cell RNA sequencing (scRNA-seq) revealed differences in conjunctival immune cell populations between B6 and Pinkie. 2A) UMAP of 19 distinct immune cell clusters in the conjunctiva generated from single-cell transcriptomic profiles of CD45⁺ cells using Seurat package v3; 2B) UMAP comparing conjunctival immune cell clusters obtained by scRNA seq of CD45⁺ cells obtained from 8 mice female C57BL/6J and 8 Pinkie age 16 weeks. Percentage of the cells in each cluster is shown in parentheses and the cell count and percentage for clusters are provided in Table 1; 2C) Heatmap of the top 50 differentially expressed genes in C57BL/6 and Pinkie conjunctival immune cells. Color of the heatmap based on the natural log of the normalized RNA expression; 2D) Violin plots showing expression of IL-17a (top) and IL-17f (bottom) with expression in each cluster in the plots to the right. *adj p value≤0.05 between strains; 2E) Violin plots of γδT17 signature genes Ltb, Cxcr6, Rorc and IL1rf that have significantly higher expression in Pinkie vs. C57BL/6 (B6). **** p≤0.0001;

FIGS. 3A-3D show increased γδ T17 cells in the Pinkie conjunctiva. 3A) Flow cytometry of γδ T cell receptor positive cells (γδ TCR) left, IL-17A⁺CD3⁺γδ TCR− center and IL-17A⁺CD3⁺γδTCR⁺ right [C57BL/6 (B6) strain top and Pinkie strain bottom) Bar graphs show mean+/−SD of percentage of cells in these groups (n=6/group); 3B) Confocal microscopy of whole mount conjunctivas obtained from B6 and Pinkie costained with antibodies specific for γδ TCR and conjunctival specific cytokeratin 13 (CK-13) (top), IL-17A (middle), and γδ T cell transcription factor RORγt (bottom) (n=3 per group). The number of γδ T cells positive for IL-17A and RORγt are shown in the bar graphs to the right. Antibody details are provided in Table 3; 3C) Confocal microscopy of whole mount conjunctivas obtained from B6 and Pinkie stained with CXCL16 antibody (left, n=3). Minimal staining was observed in the B6 conjunctival. Comparison of CXCL16 expression level in the conjunctiva measured by real-time PCR (n=6); 3D) Tear concentrations of IL-17 signature cytokines IL-12A/IL-23A (top) and TNF-α (middle), and angiogenic factor VEGF-A (bottom) measured by Luminex multiplex assay (n=5-12/group);

FIGS. 4A-4D show suppressive effects of 9-cis retinoic acid. 4A) IL-17A/F concentration in supernatants of cultured γδ T cells isolated with magnetic beads from C57BL/6 or Pinkie spleens. Cells are stimulated with anti-CD3/CD28 beads or beads plus recombinant IL-23 without or with addition of 100 nM 9-cis retinoic acid (RA). IL-17A/F is measured by ELISA. !p≤0.05 between treatment groups, * p≤0.05 between B6 and Pinkie strains; 4B) Volcano plots showing expression level of genes in monocytes cultured in media alone, media plus LPS or media plus LPS and 100 nM 9-cis RA. A mouse myeloid Innate Immunity Nanostring array was used to evaluate gene expression. Dotted vertical lines indicate less or greater than 1.5 log 2 fold change and horizontal lines indicates genes with an adjusted p value≥0.05. Red dots are genes that are significantly increased by LPS (top) or by LPS+9-cis RA (bottom). γδ T17 inducers (TNF-α, IL-1a, IL-1b and IL-23a) are stimulated by LPS and reduced by 9-cis RA; 4C) ATAC Seq: principal component analysis of peak sequences identified by ATAC seq in 3 experimental groups of cultured murine monocytes: control, cells stimulated with LPS and cells stimulated with LPS and 100 nM 9-cis RA (n=2/group); 4D) Sequence logos of transcription factor binding motifs that are found to be increased (up) or decreased (down) in the second group compared to the first group (top 4-5 motifs are shown for each group, except control vs. LPS where no decrease in motifs are found). Motifs are identified by the HOMER peak caller from databases of known motif sequences. (Yan et al., 2020);

FIGS. 5A-5C show pathway analysis. 5A) Heatmap of canonical pathways showing significant differences between strains and cell clusters was generated by Qiagen™ Ingenuity Pathway Analysis™. This analysis identified the pathways from the Ingenuity Pathway Analysis library of canonical pathways that were most relevant to the data set. Molecules from the data set that had an adjusted p value≤0.05 and were associated with a canonical pathway in the Ingenuity Knowledge Base were considered for the analysis. The significance of the association between the data set and the canonical pathway was measured in two ways: 1) A ratio of the number of molecules from the data set that map to the pathway divided by the total number of molecules that map to the canonical pathway is displayed; and 2) A right-tailed Fisher's Exact Test was used to calculate a p-value determining the probability that the association between the genes in the dataset and the canonical pathway is explained by chance alone. IL-17 signaling and PPARα/RXRα activation pathways are among the pathways identified with significant differences; 5B) IL-17 signaling pathway network showing relationship between molecules generated with Qiagen Ingenuity Pathway Analysis with modification. All connections are supported by at least one reference from the literature, from a textbook, or from canonical information stored in the Ingenuity Knowledge Base. Lines and arrows between nodes represent direct (solid) or indirect (dashed) interactions between gene products and are displayed by cellular localization (extracellular space, plasma membrane, cytoplasm, or nucleus). Rectangles are cytokines and cytokine receptors, triangles are phosphatases, concentric circles are groups or complexes, diamonds are enzymes and ovals are transcriptional regulators or modulators. P=phosphorylation, U=ubiquitination; 5C) Heatmap of differentially expressed genes in conjunctival bulk RNA seq between C57BL/6 (B6) and Pinkie strains that includes IL-17 pathway associated genes in red and other innate inflammatory mediators. All the genes passed through the Benjamini-Hochberg procedure to exclude false discovery; the selected genes had an adjusted p value≥0.05. Each row represents a specific gene, right column represents the Pinkie strain and left column represents B6;

FIGS. 6A-6E show comparison of γδT17 and dry eye signs in bone marrow chimeras. 6A) Conjunctival goblet cell number in C57BL/6 mice exposed to desiccating stress (DS) for 5 days (DS5) with or without systemic treatment with anti-IL-17 neutralizing antibody or isotype control as described in the methods herein; 6B) Representative flow cytometry plots of donor (CD45.2⁺) and recipient (CD45.1⁺) bone marrow derived cells (left) and γδ TCR high and low CD3⁺ T cells in the conjunctivas of Pepc/BoyJ recipient (host) chimeric mice reconstituted with B6 or Pinkie bone marrow after 5 days of desiccating stress. Method of chimera creation is provided in FIG. 11; 6C) Top left: bar graph shows percentage of CD45.2⁺CD3⁺γδ/TCR⁺ in the recipient conjunctiva (n=11/group). Bottom left: histogram of percentage of IL-17⁺ cells from CD45.2⁺CD3⁺γδTCR⁺ gate in representative sample. Right: mean+/−SD percentage (top) and mean fluorescent intensity (bottom) of IL-17A⁺CD3⁺γδ TCR⁺ cells in the conjunctiva of chimeras (n=11/group); 6D) Confocal microscopy of whole mount conjunctivas or cryosections obtained from B6 and Pinkie bone marrow chimeras created as shown in FIG. 3 with or without systemic treatment with anti-IL-17 neutralizing antibody or isotype control as described in the methods and exposed to DS for 5 days stained with antibody specific for MMP-9 (top), evaluated for in situ gelatinase (zymography) activity in cryosections (middle), or stained with polyclonal antibody to cornified envelope precursor SPRR2 (bottom) (n=3 per group). Bar graphs to the right show mean±SD fluorescent intensity of the fluorochrome/fluorescent gelatin measured by Nikon Elements software (n=3/group); 6E) Conjunctival goblet cell number in Pinkie donor bone marrow chimeric mice exposed to desiccating stress (DS) for 5 days (DS5) with or without systemic treatment with anti-IL-17 neutralizing antibody or isotype control as described in the methods. Representative photomicrographs of periodic acid-stained sections for each treatment group (left) and graph of mean±SD of goblet cells/mm (n=5). Some goblet cells in the control group appear entrapped in the epithelium as previously reported. (Corrales et al., 2011);

FIGS. 7A-7E show corneal neovascularization, opacification and ulceration with aging. 7A) Appearance non ulcerated (NC) C57BL/6 (B6) and NC and ulcerated (UC) Pinkie corneas in 40-50-week-old mice; 7B) Volcano plots of differentially expressed genes in corneas of NC B6 and Pinkie (left) and NC and UC Pinkie (right) detected by a mouse myeloid Innate Immunity Nanostring array. Dotted vertical lines indicate less or greater than 1.5 log 2 fold change and horizontal lines indicates genes with an adjusted p value≥0.05. Red dots are genes that are significantly increased in Pinkie NC vs. B6 NC (left) or in Pinkie UC vs. Pinkie NC (left). Labeled genes in the left plot are found in the IL-17 signaling pathway; 7C) Heat maps generated from the nanostring array of IL-17 pathway genes; 7D) Fold change of expression level of factors involved in pathogenesis of corneal vascularization (Vegfa, Tnf, Fgf7) or corneal ulceration (Mmp9) measured by RT-PCR. Values are mean mean±SD (n=4/sample); 7E) Immunostaining of blood (CD31) and lymphatic (LyVE-1) endothelial cell markers in 25- and 60-week-old B6 and Pinkie corneas. Arrows indicate corneal epithelium. n=4/sample). *p≤0.01; **p≤0.001; ****p≤0.0001;

FIG. 8 shows a summary of RXRα mediated suppression of IL-17 production by γδ T cells and IL-17 mediated dry eye disease. RXRα suppresses production of IL-17 inducers (IL-23, IL-1β and TNF-α) by myeloid cells and directly suppresses IL-17 production by activated γδ T cells. IL-17 promotes corneal barrier disruption, increased expression of the cornified envelope precursor SPRR2 that decreases epithelial lubricity and seals goblet cell openings and reduction in mucin filled conjunctival goblet cells;

FIG. 9 shows UMAP feature plots of highly expressed genes in each cluster (cluster identity in parentheses) identified in scRNA-seq, except cluster 1 where expression of Lcn2 is low;

FIG. 10 shows violin plots for γδT17 signature genes Ltb, Cxcr6, Rorc and IL1rf in cell clusters identified in scRNA-seq in Pinkie and C57BL/6 strains;

FIG. 11 shows generation of bone marrow chimeric mice. Bone marrow ablation in Pepc/BoyJ recipient (host) mice was accomplished with ¹³⁷Cs irradiation with 1300 cGy, followed by intraorbitally injection of 2×10\hat{ }6 bone marrow cells from wild type B6 or Pinkie donors. Two weeks after receiving donor cells, chimeric mice were exposed to desiccating stress for 5 days to create dry eye and presence of donor bone marrow derived cells was identified by flow cytometry performed on conjunctival samples. The representative scatter plot shows the endogenous (CD45.1) or transplanted (CD45.2) immune cells in the conjunctiva. The phenotype of CD45.2⁺ cells shown in the gate were further characterized.

FIG. 12 shows IL-17A/F cytokine concentration measured in supernatants of cultured mouse γδ T cells isolated with magnetic beads from C57BL/6 spleens.

FIG. 13 provides UMAP of 14 distinct immune cell clusters in corneas from C57B/6 (B6) and Pinkie generated from single-cell transcriptomic profiles of CD45⁺ cells using Seurat package v3.

FIG. 14 demonstrates heatmaps comparing the percentages of 14 cell types in B6 and Pinkie corneas.

FIG. 15 shows a volcano plot comparing level of differentially expressed genes in corneas of B6 and Pinkie strains.

### DETAILED DESCRIPTION

### I. Definitions

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

The terms “drop” or “microdrop” as used herein refers to a liquid formulation less than 0.1 mL. A microdrop may be approximately 0.001 mL, 0.002 mL, 0.003 mL, 0.004 mL, 0.005 mL, 0.006 mL, 0.007 mL, 0.008 mL, 0.009 mL, 0.01 mL, 0.02 mL, or 0.03 mL. A drop may be approximately 0.04 mL, 0.05 mL, 0.06 mL, 0.07 mL, 0.08 mL, or 0.09 mL.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The term “prevention” (or its grammatical equivalents) as used herein when in reference to any eye disorder, refers to intervention in an attempt to keep the eye disorder from occurring or reoccurring in an individual or to delay the onset of the eye disorder in an individual.

The term “treatment” (or its grammatical equivalents) as used herein refers to intervention in an attempt to alter the natural course of the disorder being treated. Treatment may serve to accomplish one or more of various desired outcomes, including, for example, alleviation of symptoms, diminishment of any direct or indirect pathological consequences of the disease, lowering the rate of disease progression, amelioration or palliation of the disease state, and remission or improved prognosis.

It is contemplated that any embodiment discussed in this specification can be implemented with respect to any method or composition of the disclosure, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### II. RXR Agonists

Embodiments herein encompass compositions, and methods for using the compositions, comprising at least one RXR agonist. In some embodiments, the RXR agonist comprises a composition capable of binding to a heterodimer or homodimer comprising an RXR molecule. The RXR agonist may be an RXRα agonist. In certain cases, the RXRα agonist is a small molecule. The RXR agonist may comprise 9-cis retinoic acid, oleic acid, omega-3 docosahexaenoic acid, vitamin D, bexarotene, taxerotene, honokiol, AM80, rosiglitazone, Drupanin, garcinoic acid, NEt-3IB, any analog thereof, or a combination thereof. The RXR agonist may comprise a steroid ring structure. In some embodiments, the RXR agonist comprises a retinol ring structure (including any vitamin A composition) with no chemical modifications or one or more modifications.

In some embodiments, the RXR agonist, such as 9-cis RA, for example, suppresses production of IL-17 by γδ T cells and/or IL-17 inducing cytokines by monocytes. The RXR agonist useful in embodiments herein may be screened from a library of potential RXR agonists. The screen may be any method capable of detecting RXR agonist activity. These include ligand binding assays and/or biological activity assays, as examples.

### III. Eye Disorders

Certain embodiments herein concern the treatment or prevention of one or more eye disorders in an individual and/or treatment or prevention of dry eye that is the result of a medical condition that is not an eye disorder and/or treatment or prevention of dry eye that is the result of aging, an environment, and so forth. In some embodiments, the eye disorder is treated or prevented by administering one or more RXR agonists to the individual. The eye disorder may be any disorder wherein overproduction of IL-17 by γδ T cells and/or overproduction of IL-17 inducing cytokines by monocytes occurs. The eye disorder may be a dry eye disorder, such as aqueous deficient dry eye. In some embodiments, the individual has an alteration in the Meibomian gland. In some embodiments, the individual has Meibomian gland disease. The eye disorder may be an ocular surface inflammatory condition, such as scleritis. The individual may have dry eyes because they have an eye disorder or may have dry eyes as a result of another medical condition that has dry eyes as a secondary effect (e.g., not all individuals with the medical condition have dry eyes).

In specific embodiments, the individual has decreased tear production for any reason. In specific embodiments, the individual is advanced in age, such as being (or being at least) 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years of age. The decreased tear production may come from certain medical conditions, including Sjogren's syndrome, allergic eye disease, rheumatoid arthritis, lupus, scleroderma, graft vs. host disease, sarcoidosis, thyroid disorders, diabetes, scleroderma, Parkinson's disease, Graves' disease, or vitamin A deficiency. The decreased tear production may come from certain medicines, including antihistamines, decongestants, hormone replacement therapy, antidepressants, tranquilizers, medicines for high blood pressure, acne, certain heart medications, diuretics, birth control pills and ulcer medications. The decreased tear production may come from corneal nerve desensitivity caused by contact lens use, nerve damage or laser eye surgery. The decreased tear production may come from hormonal changes in women, such as after menopause or during pregnancy.

In some embodiments, the individual has increased tear evaporation. In some cases, the oil film produced by small glands on the edge of the eyelids (meibomian glands) may be clogged. Blocked meibomian glands may occur in individuals with rosacea or other skin disorders.

In some embodiments, an individual may have increased tear evaporation from having posterior blepharitis (meibomian gland dysfunction), eye allergies, Vitamin A deficiency, exposure to preservatives, such as in topical eye drops, exposure to wind, smoke or dry air, from blinking less often (such as occurring with certain conditions, such as Parkinson's disease; or upon concentration during certain activities, such as while reading, driving or working at a computer), or from eyelid problems, such as the lids turning outward (ectropion) or the lids turning inward (entropion).

In specific embodiments, the individual has dry eye symptoms such as dry, gritty or burning sensation in the eyes, redness, watery or teary eyes, mucus that make the eyes feel “glued shut” after sleeping, the feeling of something in the eye or eyestrain, itching, light sensitivity may also occur. In certain embodiments the symptoms are worse later in the day. In specific embodiments, dry eye can be diagnosed based on symptoms. In specific embodiments, one or more tests are utilized for diagnosis, such as measuring tear production, special dyes, and evaluation of the constitution of the tear film. In certain aspects the tests exclude other potential problems, such as conjunctivitis, that can produce the same symptoms.

In some embodiments, an individual is at risk for dry eye higher than the general population, such as being older than 50, being a woman (such as with pregnancy, using birth control pills or during menopause), eating a diet that is low in vitamin A, eating a diet that is low in omega-3 fatty acids, wearing contact lenses, and/or having a history of refractive surgery.

### IV. Pharmaceutical Compositions

In certain aspects, the compositions or agents for use in the methods, such as any RXR agonist, are suitably contained in a pharmaceutically acceptable carrier. In some embodiments, the carrier is non-toxic, biocompatible and is selected so as not to detrimentally affect the biological activity of the agent. The agents in some aspects of the disclosure may be formulated into preparations for local delivery (i.e. to a specific location of the body, such as any eye tissue or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, hyaluronic acid, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, dimethyl sulfoxide (DMSO), ethanol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### A. Formulations

In some embodiments, the at least one of the active compounds disclosed herein is formulated into a sustained release vehicle. The sustained release vehicle may be suitable for an eye drop, including a microdrop, and/or an injection. The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

A pharmaceutical composition can include a solvent or dispersion medium containing, for example, water, ethanol, polyol (for example, glycerol, propylene glycol, and liquid polyethylene glycol, and the like), DMSO, suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. Alternatively, administration may be by orthotopic, intraocular, subconjunctival, intradermal, subcutaneous, intramuscular, intraperitoneal, or intranasal administration. In some embodiments, administration of the compositions occurs via an eyedrop, which may be a microdrop, drop, cream, or ointment. In some embodiments, the compositions occurs by an ointment or cream delivered to the eye or eyelid. Any such compositions described herein would normally be administered as pharmaceutically acceptable compositions that include physiologically acceptable carriers, buffers or other excipients.

Upon formulation, solutions may be administered in a manner compatible with the dosage formulation and in such amount as is therapeutically or prophylactically effective. The formulations are easily administered in a variety of dosage forms, such as the type of injectable solutions described above.

### B. Other Agents

It is contemplated that other agents may be used in combination with certain aspects of the present embodiments to improve the therapeutic efficacy of treatment. These additional agents include corticosteroids, cyclosporine A, lifitegrast, tetracyclines (doxycycline, minocycline) and varenicline, and/or agents that may help manage, prevent, or treat any disorder disclosed herein.

### V. Administration of Therapeutic Compositions

In some embodiments, therapeutic compositions, which may also be referred to as pharmaceutical compositions, are administered to a subject. Different aspects may involve administering an effective amount of a composition to a subject. In some embodiments, an antibody or antigen binding fragment capable of binding to RXR (or any dimer of RXR) may be administered to the subject to protect against or treat a condition (e.g., an eye disorder). Alternatively, an expression vector encoding one or more such antibodies or polypeptides or peptides may be given to a subject as a preventative treatment. Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The therapy provided herein may comprise administration of a combination of therapeutic agents, such as a first therapy and a second therapy. The therapies may be administered in any suitable manner known in the art. For example, the first and second treatment may be administered sequentially (at different times) or concurrently (at the same time). In some embodiments, the first and second treatments are administered in a separate composition. In some embodiments, the first and second treatments are in the same composition.

In some embodiments, the first therapy and the second therapy are administered substantially simultaneously. In some embodiments, the first therapy and the second therapy are administered sequentially. In some embodiments, the first therapy, the second therapy, and a [third] therapy are administered sequentially. In some embodiments, the first therapy is administered before administering the second therapy. In some embodiments, the first therapy is administered after administering the second therapy.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the therapy is administered intravenously, intramuscularly, subcutaneously, topically, orally, subconjunctivally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, the therapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 μg/kg or mg/kg. In some embodiments, the therapy is administered at a dose between 0.14 mg/kg to 0.57 mg/kg.

In some embodiments, a single dose of the second therapy is administered. In some embodiments, multiple doses of the second therapy are administered. In some embodiments, the second therapy is administered at a dose of between 1 mg/kg and 100 mg/kg. In some embodiments, the second therapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 μg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 0.1 nM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of ng/kg, μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In some embodiments, the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—General Embodiments

Embodiments of the present disclosure including methods to investigate the mechanism for developing dry eye disease, such as in the Pinkie mouse strain with a loss of function RXRα mutation.

In some embodiments, measures of dry eye disease were assessed in the cornea and conjunctiva. Expression profiling by single-cell RNA sequencing (scRNA-seq) was performed to compare gene expression in conjunctival immune cells. Conjunctival immune cells were immunophenotyped by flow cytometry and confocal microscopy. Activity of RXRα ligand 9-cis retinoic acid (RA) was evaluated in cultured monocytes and γδ T cells.

Analysis of certain methods herein revealed, compared to wild type (WT) C57BL/6, Pinkie has increased signs of dry eye disease, including corneal barrier disruption, conjunctival cornification and goblet cell loss, and corneal vascularization, opacification, and ulceration with aging. scRNA-seq of conjunctival immune cells identified γδ T cells as the predominant IL-17 expressing population in both strains and there is a 4-fold increased percentage of γδ T cells in Pinkie. Compared to WT, significantly increased expression of IL-17a and IL-17f in conventional T cells and IL-17f in γδ T cells was found in Pinkie. Flow cytometry and immunostaining revealed an increased number of IL-17⁺ γδ T cells in Pinkie. Tear concentration of the IL-17 inducer IL-23 is significantly higher in Pinkie. 9-cis RA treatment suppresses stimulated IL-17 production by γδ T and stimulatory activity of monocyte supernatant on γδ T cell IL-17 production. Compared to WT bone marrow chimeras, Pinkie chimeras have increased IL-17⁺ γδ T cells in the conjunctiva after desiccating stress and anti-IL-17 treatment suppresses dry eye induced corneal MMP-9 production/activity and conjunctival goblet cell loss.

The findings herein indicate that RXRα suppresses generation of dry eye disease inducing γδ T17 cells in the conjunctiva and identifies RXRα as a therapeutic target in dry eye.

### Example 2—Materials and Methods for Certain Embodiments

### Animals

The animal protocol for this study was designed according to the ARVO Statement for the use of Animals in Ophthalmic and Vision Research and was approved by the Institutional Animal Care and Use Committee at Baylor College of Medicine (Protocol AN-2032). Female C57BL/6J (B6) mice and Pepc/BoyJ aged 6-8 weeks were purchased from Jackson Laboratories (Bar Harbor, ME). The RXRα Pinkie mutant strain was purchased from the Mutant Mouse Resource and Research Centers (MRRC, University of California, Davis, Sacramento, CA) for establishing breeder colonies that were expanded in Baylor College of Medicine vivarium and refreshed and genotyped every 8 generations. At the time of the experiments, both B6 and Pinkie strains were 16-60 weeks of age and had been housed in the normal vivarium environment.

### Corneal Barrier Function Assessment

Corneal epithelial permeability to 70 kDa Oregon-Green-conjugated dextran (OGD; Invitrogen, Eugene, OR) was assessed as previously described (Alam et al., 2020b). Briefly, 1 μL of OGD (50 mg/mL) was instilled onto the ocular surface 1 min before euthanasia; the eye was then rinsed with 2 mL phosphate-buffered saline (PBS) from the temporal and nasal side and photographed with a high-resolution digital camera (Coolsnap HQ2; Photometrics, Tucson, AZ) attached to a stereoscopic zoom microscope (SMZ 1500; Nikon, Melville, NY), under fluorescence excitation at 470 nm. The severity of corneal OGD staining was graded in digital images using NIS Elements (version 3.0; Nikon) within a 2-mm diameter circle placed on the central cornea by 2 masked observers. The mean fluorescence intensity measured by the software inside this central zone was transferred to a database, and the results were averaged within each group.

### Measurement of Goblet Cell Density

Following euthanasia, eyes and ocular adnexa were excised from B6 and Pinkie mice (n=5/group) and the tissues were fixed in 10% formalin followed by paraffin embedding, 5-μm sections were cut with a microtome (Microm HM 340E; Thermofisher Wilmington, DE) and stained with periodic acid Schiff (PAS) reagent. Sections from both eyes in each group were examined and photographed with a microscope (Eclipse E400; Nikon) equipped with a digital camera (DXM1200; Nikon) Using the NIS Elements software; goblet cells were manually counted. To determine the length of the conjunctival goblet cell zone, a line was drawn on the surface of the conjunctiva image from the first to the last PAS⁺ goblet cell. Results are presented as PAS⁺ goblet cells/mm.

### RNA Isolation and Quantitative PCR

Following euthanasia, the cornea/conjunctiva was excised and total RNA was extracted using an RNeasy® Plus Mimi Kit (Cat No. 74134, QIAGEN GmbH, Hilden, Germany) according to manufacturer's instruction. The RNA concentration was measured, and cDNA was synthesized using the Ready-To-Go-You-Prime-First-Strand kit (GE Healthcare). Quantitative real-time PCR was performed with specific probes Murine MGB probes, Cxcl16 (Mm00801778), Sprr2a (Mm00845122_s1), Sprr2f (Mm00448855_s1), Sprr2g (Mm01326062_m1), Vegfa (Mm00437304), Vegfb (Mm00442102), Vegfc (Mm00437310), Tnf (Mm00443260), Fgf7 (Mm00433291), Mmp9 (Mm00442991) and hypoxanthine phosphoribosyl transferase (Hprt1, Mm00446968). The Hprt-1 gene was used as an endogenous reference for each reaction. The results of real-time PCR were analyzed by the comparative CT method, the CT value of Pinkie were compared to that of B6.

### Tear Washings and Multiplex Immunoassay

Tear-fluid washings were collected from both mouse strains using capillary tubes as previously described (Zheng et al., 2010), and cytokine concentrations in tear samples were assayed using a commercial ProcartaPlex Luminex Assay according to the manufacturer's protocol (Thermofisher). The reactions were detected with streptavidin-phycoerythrin using a Luminex LX200 (Austin, TX, USA) (Zaheer et al., 2018). One sample consisted of tear washings from both eyes of 4 mice pooled (8 μL) into a tube containing 8 μL of PBS+0.1% BSA and stored at −80° C. until the assay was performed. Results are presented as the mean±standard deviation (picograms per milliliter).

### Flow Cytometry and Cell Sorting

Conjunctivae were excised, chopped with scissors into tiny pieces, and incubated with 0.1% type IV Collagenase for 1 hour to yield single-cell suspensions. Samples were incubated with anti-CD16/32 (2.4G2, Catalog no. 553141, BD Pharmingen™, San Diego, CA), for 5 minutes at room temperature and subsequently stained with anti-CD45 (clone 30-F11, Catalog no. 103138, BioLegend) and with an infra-red fluorescent viability dye (Life Technologies, Grand Island, NY). The gating strategy was as follows: lymphocytes were identified by forward-scatter area (FSC-A) and side scatter area (SSC-A) gates, followed by two singlets gates (FSC-A vs. FSC-W and SSC-A vs. SSC-W) followed by live/dead identification using the infra-red fluorescent viability dye. The CD45+ cells were sorted using the Aria-II cell sorter at the Baylor College of Medicine cytometry and cell sorting core.

Antibodies for phenotyping IL-17⁺ cells in the conjunctiva included: anti-CD45 (clone 30-F11, Catalog no. 103138, BioLegend), Alexa Fluor®488 anti-mouse CD45.1 (Clone A20, catalog #110718, BioLegend Way San Diego, CA), Brilliant Violet 510™ anti-mouse CD45.2 (Clone 104, catalog #109838, BioLegend Way San Diego, CA), PerCP/Cyanine5.5 anti-mouse CD3ε (Clone 500A2, catalog #152312, BioLegend Way San Diego, CA), PE Anti-Mouse γδ T-Cell Receptor (Clone GL3, catalog #553178, BD Pharmingen™, San Diego, CA), Alexa Fluor®647 anti-mouse IL-17A (Clone TC11-18H10, catalog #560184, BD Pharmingen™, San Diego, CA). A violet live/dead fixable dye (Life Technologies) was used to exclude dead cells. A Canto II flow cytometer (BD Biosciences) and FlowJo 7.6.5 software (TreeStar, Ashland, OR, USA) were used for analysis.

### Library Preparation

Single-cell gene expression libraries were prepared using the Chromium Single Cell Gene Expression 3v3.1 kit (10× Genomics) at the Single Cell Genomics Core at Baylor College of Medicine. In brief, single cells, reverse transcription (RT) reagents, Gel Beads containing barcoded oligonucleotides, and oil were loaded on a Chromium controller (10× Genomics) to generate single-cell Gel Beads-In-Emulsions (GEMs) where full-length cDNA was synthesized and barcoded for each single cell. Subsequently the GEMs are broken and cDNA from every single cell is pooled. Following cleanup using Dynabeads MyOne Silane Beads (Thermofisher, Waltham, MA), cDNA is amplified by PCR. The amplified product is fragmented to optimal size before end-repair, A-tailing, and adaptor ligation. The final library was generated by amplification.

### Sequencing of 10× GEM 3′v3.1 Single Sell Libraries

The BCM Genomic and RNA Profiling (GARP) Core initially conducted sample quality checks using the NanoDrop spectrophotometer and Agilent Bioanalyzer 2100. To quantitate the adapter-ligated library and confirm successful P5 and P7 adapter incorporations, the Applied Biosystems ViiA7 Real-Time PCR System and a KAPA Illumina/Universal Library Quantification Kit (p/n KK4824) was used. The GARP core sequenced the libraries on the NovaSeq 6000 Sequencing System using the S2 v1.0 Flowcell as follows. Cluster Generation by Exclusion Amplification (ExAMP): Using the concentration from the ViiA7™ qPCR machine above, 150 μM of the equimolar pooled library was loaded onto one lane of the NovaSeq S2 v1.0 flowcell (Illumina p/n 20012860) following the XP Workflow protocol (Illumina kit p/n 20021664) and amplified by exclusion amplification onto a nanowell-designed, patterned flowcell using the Illumina NovaSeq 6000 sequencing instrument. PhiX Control v3 adapter-ligated library (Illumina p/n FC-110-3001) was spiked-in at 1% by weight to ensure balanced diversity and to monitor clustering and sequencing performance. The libraries were sequenced according to the 10× Genomics protocol, 28 cycles for Reads 1, 10 cycles each for the i7 and i5 reads, and 90 cycles for Read 2. An average of 251 million read pairs per sample was sequenced. FastQ file generation was executed using bcl2fastq and QC reports were generated using CellRanger v5.0.1 by the BCM Multiomics Core.

Bioinformatic Analysis of scRNA-seq Data

Raw sequence reads in the FASTQ format were aligned to the mouse reference genome using Cell Ranger Count v6.0.1 pipeline (https://cloud.10xgenomics.com) with the default settings for alignment, barcode assignment, and UMI counting of the raw sequencing data with genome reference Mouse (mm10) 2020-A. The resulting gene expression matrix was subjected to preprocessing following the guideline provided by Seurat v4.0.5. Briefly, single cells with fewer than 200 genes were filtered to remove empty droplets. The genes that were expressed in less than 3 cells in the data were. Next, a global-scaling normalization method is employed using the Seurat function “LogNormalize” that normalizes the feature expression.

### Clustering, Visualization and Cell Annotation

First, the “FindVariableFeatures” function is used to identify a set of 2000 genes that are highly variable in the two data sets, and the “FindIntegrationAnchors” and “IntegrateData” functions combined the two data sets for downstream analysis such as dimensionality reduction and clustering. Principal Components Analysis (PCA) is then performed to construct a linear dimensionality reduction of the dataset and identified the 19 PCs that contain most of the complexity of the dataset. The cells were clustered in a graph-based approach within PCA space, and then non-linear dimensionality reductions were applied using UMAP for further visualization purposes. A set of canonical cell type markers is used to assign annotation to each cluster using the Cluster Identity Predictor (CIPR) web-based tool (https://aekiz.shinyapps.io/CIPR/). Finally, differential expression was performed using the “FindAllMarkers” function in Seurat to find cluster-specific marker genes.

### Monocyte Purification and In Vitro Stimulation

Monocytes were purified from 3 days cultured mouse bone marrow cells using the monocytes isolation kit, according to the manufacturer's instruction (BM, Miltenyi Biotec, Bergisch Gladbach, Germany). 5×10⁵ monocytes plated in a 48 well plate were preincubated with 100 nM 9-cisRA for 1 hour followed by stimulation with 0.5 μg/ml LPS for 4 hours for RNA or overnight for cytokines. The total RNA was extracted using an RNeasy® Plus Mimi Kit (Cat No. 74134, QIAGEN GmbH, Hilden, Germany) according to manufacturer's instruction. The RNA and collected supernatants were stored at −80° C. until further use.

### γδT17 Cell Isolation and In Vitro Experiments

Pooled γ/δ T17 cells from the spleens of 8-10 week old B6 and Pinkie mice were isolated using the TCR γ/δ T cells Isolation Kit according to the manufacturer's instruction (Miltenyi Biotec, Bergisch Gladbach, Germany). To determine the effect of 9CisRA and monocytes conditional media on IL17 cytokine production, the purified γδ T17 cells were stimulated with anti-CD3/CD28 Dynabeads (Catalog #11452D, Life Technologies AS, Norway) alone or in combination with IL-23 (10 ng/ml, eBioscience), 9-cisRA (100 nM) or monocyte conditioned media for 96 hours for cytokine measurement.

### IL-17 ELISA

Mouse IL17 was measured from cell cultured supernatant after 96 hours incubation using a mouse IL-17 DuoSet Enzyme-linked immunosorbent assay (ELISA) (R&D systems, Minneapolis, USA)

NanoString nCounter Gene Expression Analysis

This was performed by the Genomic and RNA Profiling Core at Baylor College of Medicine using the the NanoString Technologies nCounter Gene Expression Mouse Myeloid Innate Immunity V2 Panel codeset (NS_MM_Myeloid_V2.0) containing 770 unique pairs of 35-50 bp reporter probes and biotin-labeled capture probes, including internal reference controls (NanoString, Seattle, WA) as previously described. (Alam et al., 2021a). Data was analyzed by ROSALIND® (https://rosalind.bio/), with a HyperScale architecture developed by ROSALIND, Inc. (San Diego, CA).

### Bulk RNA Seq and Data Analysis

Conjunctival epithelium was excised from B6 and Pinkie strains and total RNA was extracted using a QIAGEN RNeasy Plus Micro RNA isolation kit (Qiagen) according to manufacturer's instruction. The concentration and purity of RNA was assessed using a NanoDrop 1000 (ThermoFisher Scientific, Walhham, MA). RNA-Seq was performed by the Beijing Genomics Institute (BGI) using the BGISEQ500RS to generate 100-bp paired-end reads. The raw data were cleaned by removing reads containing adapter or poly-N sequences, and reads of low quality using SOAPnuke (version 1.5.2, parameters: −l 15 −q 0.2 −n 0.05). and the expression levels of the resulting genes and transcripts were determined using RSEM (version 2.2.5, default parameters). Detection of DEGs (differentially expressed genes) was performed with DEseq2 (Parameters: Fold Change≥2.00 and Adjusted P value≤0.05). A total of 19,511 genes were obtained as raw data. Genes were passed through the Benjamini-Hochberg procedure to obtain the critical value for false discovery and a total of 1375 genes passed with a P-value>0.0006. The selected genes IL-17 signaling pathway were clustered in a heat map.

### ATAC Seq

Cultured bone marrow derived monocytes were harvested and frozen in culture media containing FBS and 5% DMSO. Cryopreserved cells were sent to Active Motif (Carlsbad, CA) to perform the ATAC-seq assay. The cells were then thawed in a 37° C. water bath, pelleted, washed with cold PBS, and tagmented as previously described (Buenrostro et al., 2013), with some modifications. (Corces et al., 2017) Briefly, cell pellets were resuspended in lysis buffer, pelleted, and tagmented using the enzyme and buffer provided in the Nextera Library Prep Kit (Illumina, San Diego, CA). Tagmented DNA was then purified using the MinElute PCR purification kit (Qiagen, Germantown, MD), amplified with 10 cycles of PCR, and purified using Agencourt AMPure SPRI beads (Beckman Coulter, Brea, CA). Resulting material was quantified using the KAPA Library Quantification Kit for Illumina platforms (KAPA Biosystems, St Louis, MO), and sequenced with PE42 sequencing on the NextSeq 500 sequencer (Illumina).

Analysis of ATAC-seq data was similar to the analysis of ChIP-Seq data. Reads were aligned using the BWA algorithm (mem mode; default settings). Duplicate reads were removed, only reads mapping as matched pairs and only uniquely mapped reads (mapping quality>=1) were used for further analysis. Alignments were extended in silico at their 3′-ends to a length of 200 bp and assigned to 32-nt bins along the genome. The resulting histograms (genomic “signal maps”) were stored in bigWig files. Peaks (accessible regions) were identified using the MACS 2.1.0 algorithm at a cutoff of p-value 1e-7, without control file, and with the -nomodel option. Peaks that were on the ENCODE blacklist of known false ChIP-Seq peaks were removed. Signal maps and peak locations were used as input data to Active Motifs proprietary analysis program, which creates Excel tables containing detailed information on sample comparison, peak metrics, peak locations and gene annotations. For differential analysis, reads were counted in all merged peak regions (using Subread), and the replicates for each condition were compared using DESeq2. The position and frequency of motif sequences in each peak region were identified with the search tool HOMER or known sequences in databases. (Yan et al., 2020)

### Qiagen Gene Pathway Analysis

Briefly, differentially expressed genes from single cell RNA seq data were first uploaded into Qiagen's Ingenuity Pathway Analysis (IPA) system for core analysis. Analysis was performed with experimental false discovery rate of ≥0.05. Comparison analysis tool were used to identify the most relevant canonical pathways enriched in Pinkie and presented as heatmap. IL17 signaling pathway was adopted from IPA with some modification.

### Creation of Bone Marrow Chimeras

CD45.2⁺ bone marrow chimeras using bone marrow cells obtained from 12-16 week B6 and Pinkie strains were created in 6-8 week old CD45.1⁺ Pepc/BoyJ strain as previously reported. (Gibson et al., 2015; Alam et al., 2021a) Ten days after bone marrow reconstitution, mice were subjected to 5 days of desiccating stress (DS5) and T cell populations in the conjunctiva were analyzed by flow cytometry.

### Desiccating Stress and IL-17 Neutralization

DS was induced by inhibiting tear secretion with scopolamine hydrobromide (Greenpark, Houston) in drinking water (0.5 mg/mL) and housing in a cage with a perforated plastic screen on one side to allow airflow from a fan placed 6 inches in front of it for 16 h/day for 5 consecutive days. Room humidity was maintained at 20-30%. Control mice were maintained in a non-stressed (NS) environment at 50-75% relative humidity without exposure to an air draft. Mice were treated i.p. every two days with 100 μg/mouse of anti-IL-17A (Clone 17F3; BioXcell) or mouse IgG1 isotype control (Clone MOPC-21; BioXcell) starting on day −2 for the duration of DS. After 5 days of DS, mice were euthanized and immune cells were harvested from the conjunctiva for flow cytometry (n=11), eyes were embedded in paraffin for sectioning (n=5) or in optimum cutting temperature (OCT) compound (Thermofisher) for cryosectioning (n=3), or corneas were prepared for whole-mount immunostaining (n=3).

### Immunofluorescence Staining and Confocal Microscopy

The conjunctival and corneal tissue samples were dissected from female C57BL/6J mice (age 16 weeks) and fixed in 100% methanol for 20 minutes at −20° C. followed by washing with Hanks' buffered saline solution (HBSS) for 3×5 min with gentle shaking at room temperature (RT). Tissues were permeabilized with 0.4% Triton X-100 in HBSS for 30 minutes at RT and gentle shaking. 20% goat serum (Sigma, USA) diluted in HBSS was used for 1 hour blocking at RT. Subsequently, the conjunctival tissue samples were incubated with primary antibodies (Table 2) diluted in 5% goat serum in HBSS at the mentioned concentrations overnight at 4° C. with gentle shaking at dark. The samples were then washed with 0.4% Triton X-100 for 3×6 min at RT with gentle shaking, followed by incubation with secondary antibodies (Table 2) diluted in 5% goat serum/HBSS for 1 hour at RT with gentle shaking and light protection. The samples were then washed for 3×10 min with 0.4% Triton X-100 in HBSS and Hoechst (1:500 in HBSS) was added for nuclei staining (30 min at RT and dark with gentle shaking). The samples were washed 3×5 min with HBSS, mounted on slides, and flattened with coverslips. Immunofluorescence staining in whole-mount conjunctival tissue samples was visualized using laser scanning Nikon confocal microscope (Nikon A1 RMP, Nikon, Melville, NY, USA) and 0.5 μm Z-step. The captured images were processed using NIS Elements Advanced Research (AR) software version 4.20 (Nikon).

### In Situ Zymography

In situ zymography was performed to localize the gelatinase activity in corneal cryosections using a previous reported method. (De Paiva et al., 2006b) Sections were thawed and incubated overnight with reaction buffer, 0.05 M Tris HCl, 0.15 M NaCl, 5 mM CaCl₂), and 0.2 mM NaN₃, pH 7.6, containing 40 mg/ml FITC-labeled DQ gelatin, which was available in a gelatinase/collagenase assay kit (EnzChek, Thermofisher). As a negative control, 50 mM 1,10-phenanthroline, a metalloproteinase inhibitor, was added to the reaction buffer before applying the FITC-labeled DQ gelatin to frozen sections. Proteolysis of the FITC-labeled DQ gelatin substrate yields cleaved gelatin-FITC peptides that are fluorescent at sites of net gelatinolytic activity. After incubation, the sections were washed three times with PBS for 5 min, counterstained with Hoechst 33342 dye and a coverslip was applied. Areas of gelatinolytic activity of MMPs were viewed and imaged.

### Statistical Analysis

Based on normality, parametric student T or nonparametric Mann-Whitney U tests were performed for statistical comparisons with an alpha of 0.05 using GraphPad Prism 9.0 software (GraphPad Software, Inc., San Diego, CA, USA).

### Example 3—Keratoconjunctivitis Develops in the Pinkie Strain with Reduced Rxrα Signaling

Du et al. reported the Pinkie mouse strain, with a loss of function RXRα mutation (I273N) (Du et al., 2005) that alters ligand binding and heterodimerization resulting in a 90% decrease in ligand-inducible transactivation, develops signs of dry eye with aging, but the study did not evaluate the ocular surface disease and immunopathology (Du et al., 2005). Corneal epithelial barrier disruption, loss of conjunctival goblet cells and increased expression of cornified envelope precursors by the surface epithelium are well characterized pathological features of dry eye disease (De Paiva et al., 2006a; Corrales et al., 2011).

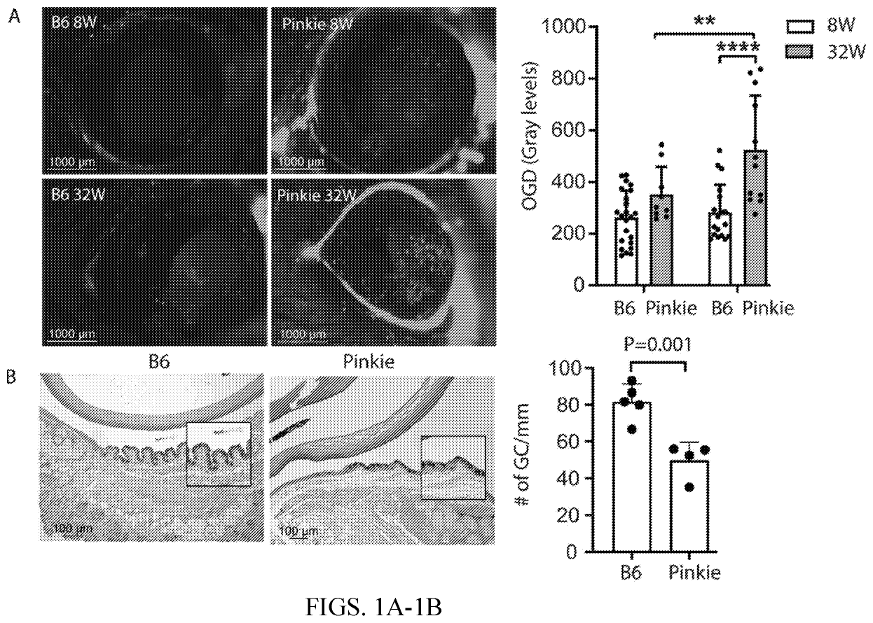

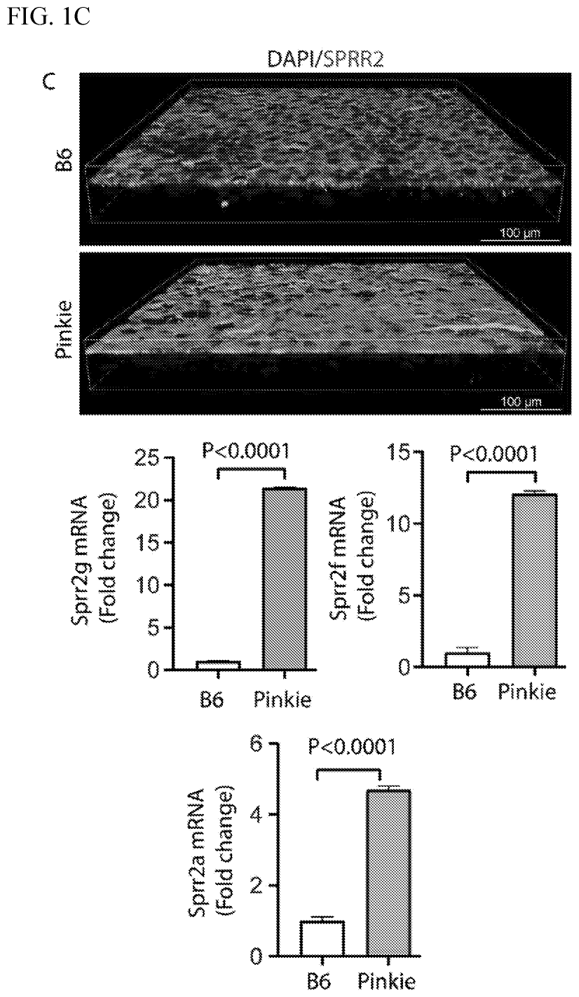

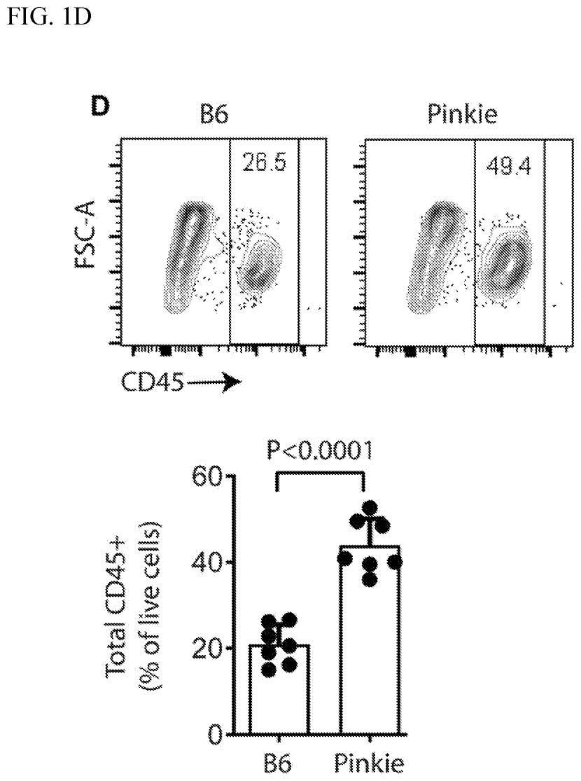

Corneal staining after topically applied 70 kDa Oregon Green Dextran (OGD) increases with corneal barrier disruption in dry eye. There is no difference in corneal OGD permeability between younger (8 W old) Pinkie and wild type (WT) C57BL/6 (B6) but OGD staining is significantly increased in 32 week old Pinkie (FIG. 1A). Reduction in conjunctival goblet cell number is another marker of dry eye. Pinkie has a significantly reduced number of PAS-positive conjunctival goblet cells at 8 weeks of age, compared to the WT strain (FIG. 1B). Increased immunoreactivity to the cornified envelope precursor SPRR2 in the conjunctival epithelium (FIG. 1C, left) in Pinkie is accompanied by increased expression of several Sprr isoform genes in the conjunctiva [Sprr2g (>20 fold), Sprr2f (>10 fold) and Sprr2a (>4fold) compared to B6 (FIG. 1C, right and below). Ocular surface desiccation stimulates recruitment of immune cells into the conjunctiva and Pinkie has significantly great number of CD45⁺ immune cells in the conjunctiva by flow cytometry (FIG. 1D). These findings indicate Pinkie has dry eye associated pathological changes in the corneal and conjunctival epithelia and the corneal epithelial disease worsens with age.

### Example 4—Pinkie has Increased γδT17 Cells in the Conjunctiva

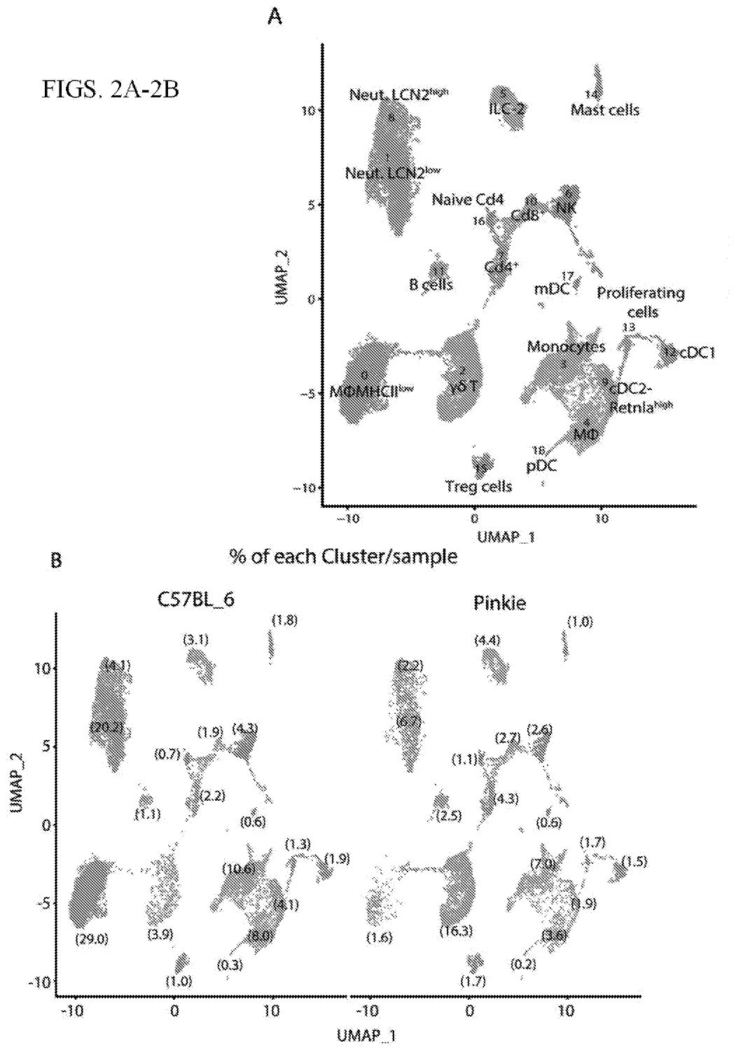

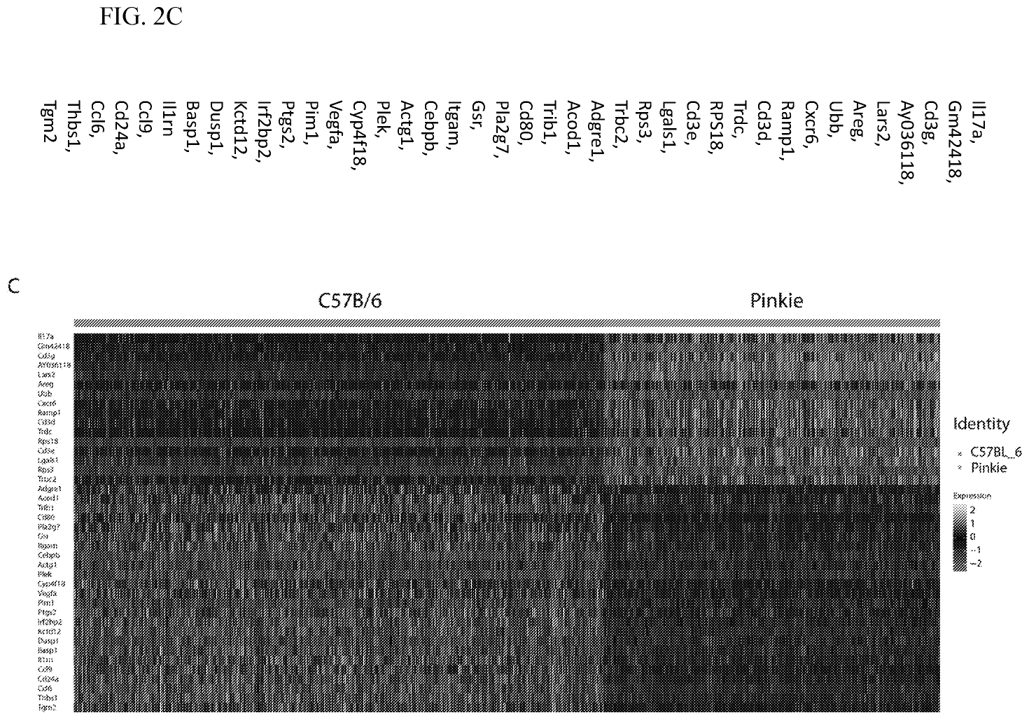

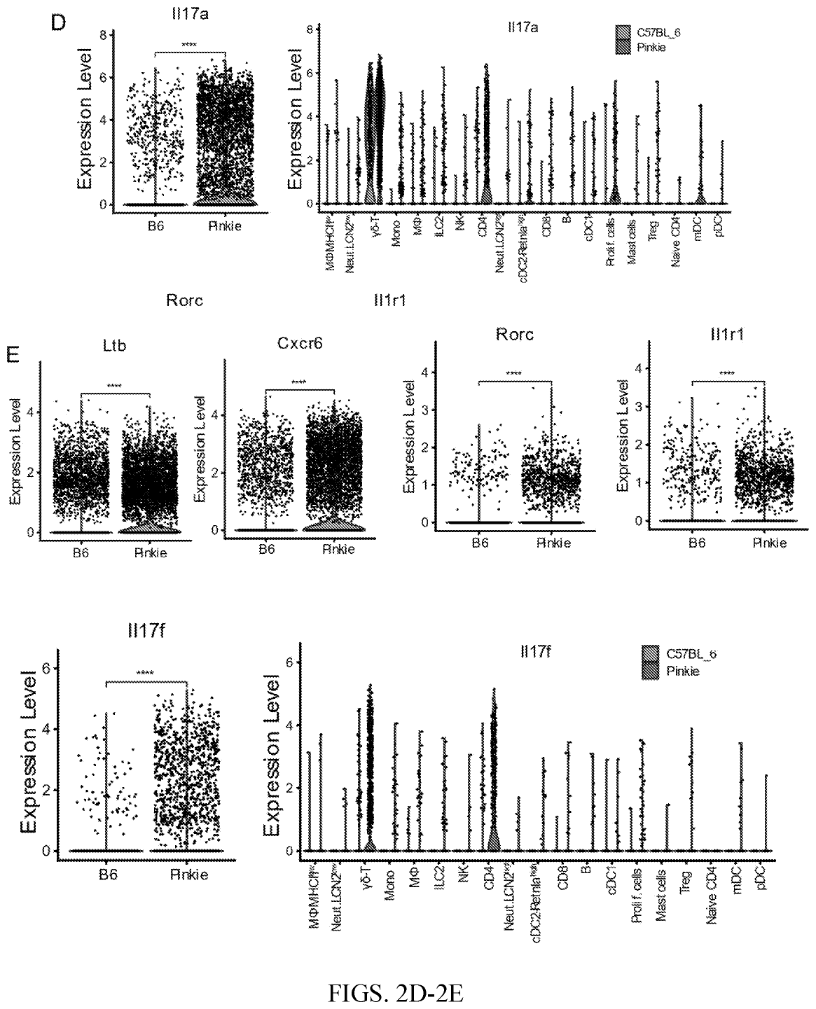

Inflammation has been found to cause ocular surface epithelial disease in dry eye. droplet-based single-cell RNA sequencing (scRNA-seq) was performed as an unbiased approach to compare immune cell types in the conjunctiva of WT and Pinkie strains. A scRNA-seq library was constructed from CD45⁺ immune cells sorted from conjunctivas of normal WT and Pinkie (n=8 biological replicates/strain) and obtained transcriptomic profiles of these cells using the 10× Genomics platform. The scRNA-seq data analysis was performed using Seurat v3. After quality assessment, filtering standard pre-processing, and doublet exclusion, a total of 11165 cells from B6 and 7096 cells from Pinkie with 2000 variable features were analyzed. Graph-based clustering using Seurat divided the cells into 19 clusters (FIG. 2A) that were identified based on the expression of signature marker genes listed in Table 1 and shown in FIG. 9. The top 20 differentially expressed genes in each cluster are listed in Table 3. The major differences between the two strains is a decreased percentage of macrophages in cluster 0 and increased percentage of 18 T cells in cluster 2 (FIG. 2B, Table 1). A heatmap of the top 50 differentially expressed genes between the strains is shown in FIG. 2C. Il17a is the top differentially expressed gene. Violin plots in FIG. 2D show γδ T cells and conventional T cells had the highest expression of both Il17a and IL17f in both strains. Pinkie has increased expression of IL17a and IL17f in conventional T cells and increased expression IL17f in γδ T cells.

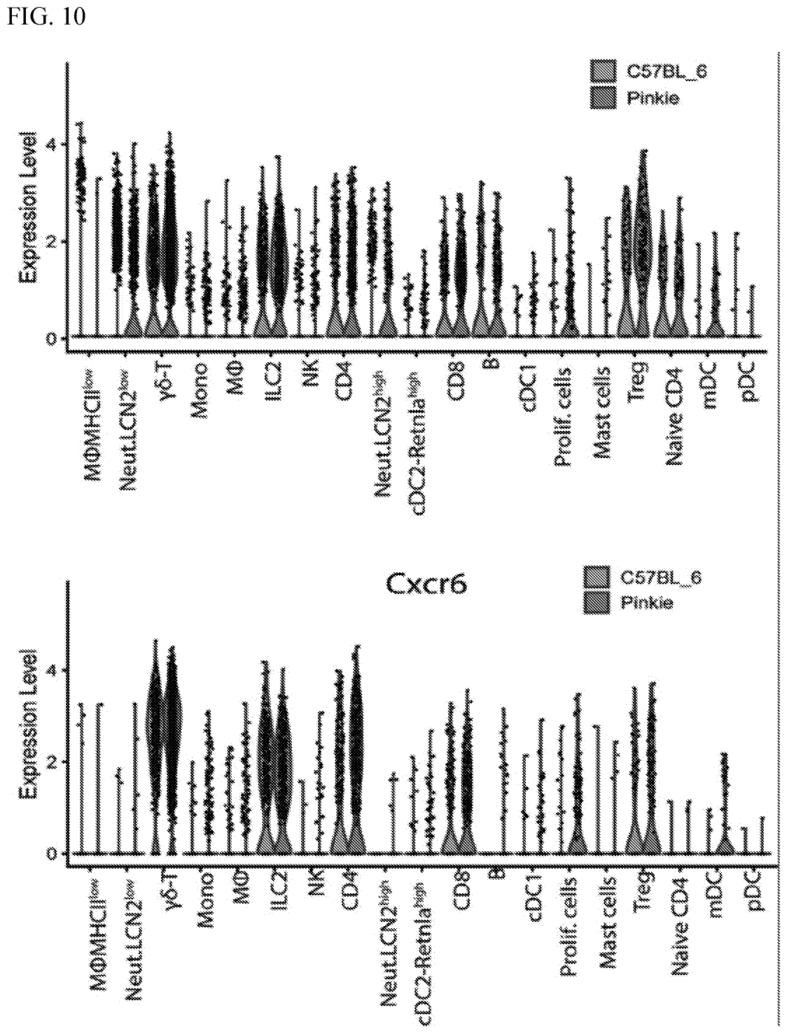

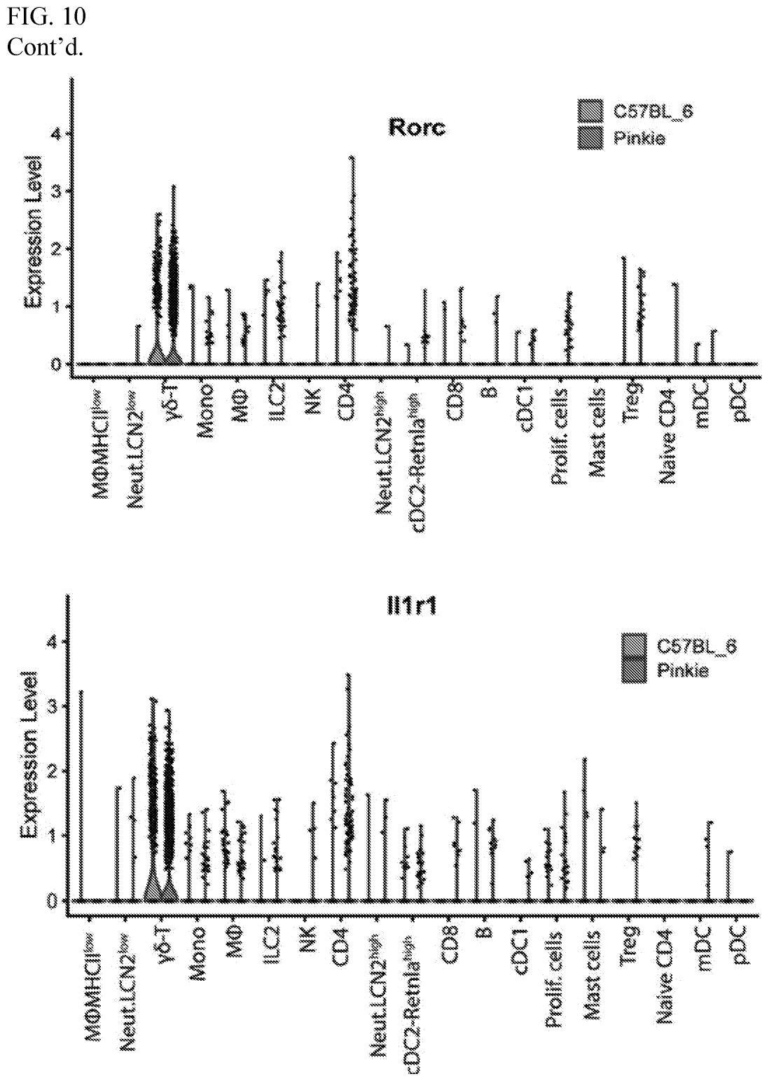

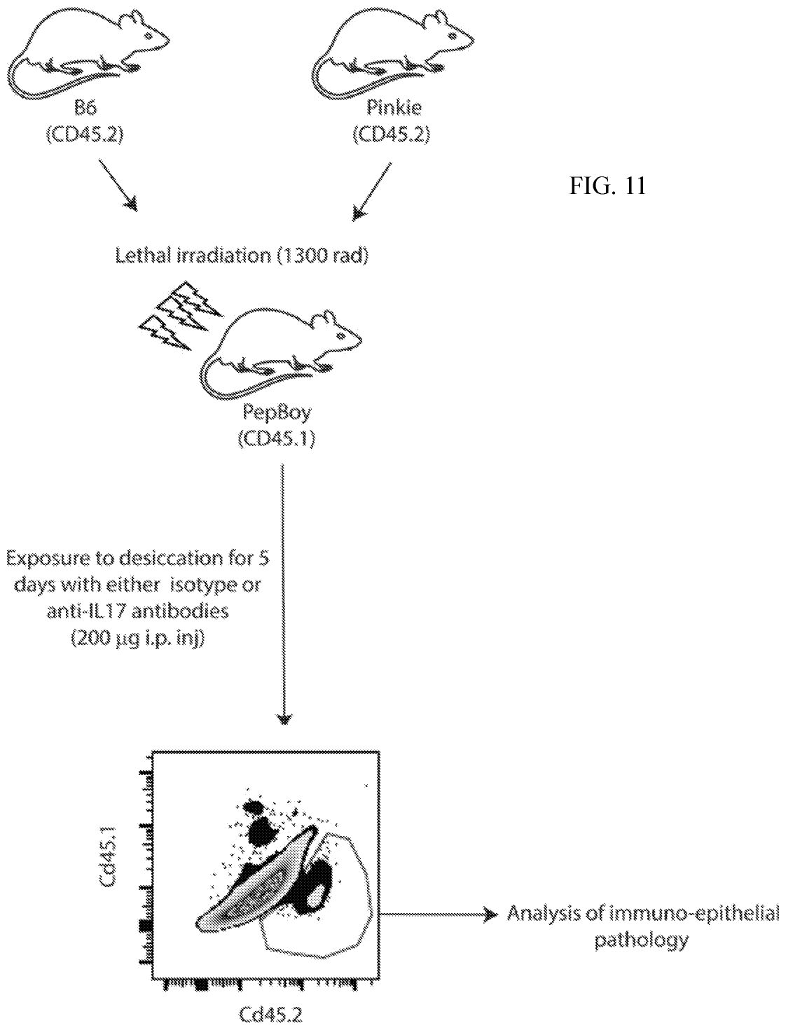

Significant between strain differences are also seen for expression of γδT17 signature genes (FIG. 2E and FIG. 10), including Ltb (Powolny-Budnicka et al., 2011), Cxcr6 (Butcher et al., 2016), Rorc (Powolny-Budnicka et al., 2011; Malhotra et al., 2013; Zuberbuehler et al., 2019) and Il1r1 (Duan et al., 2010).

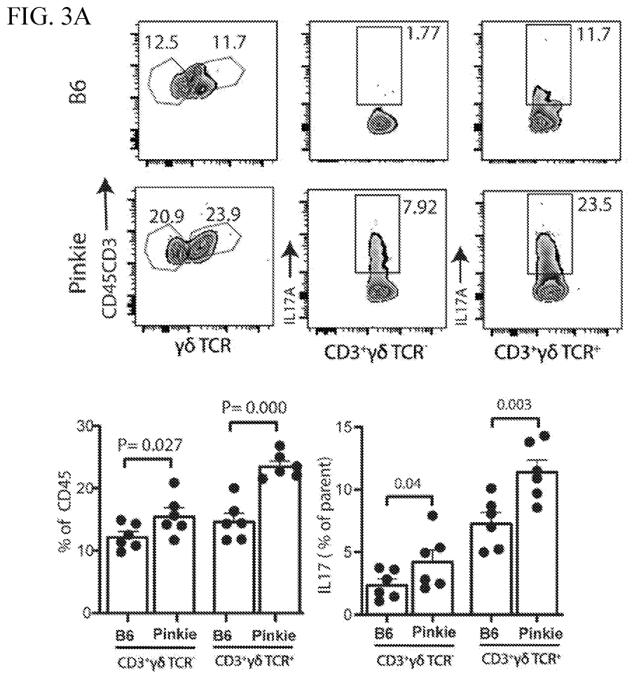

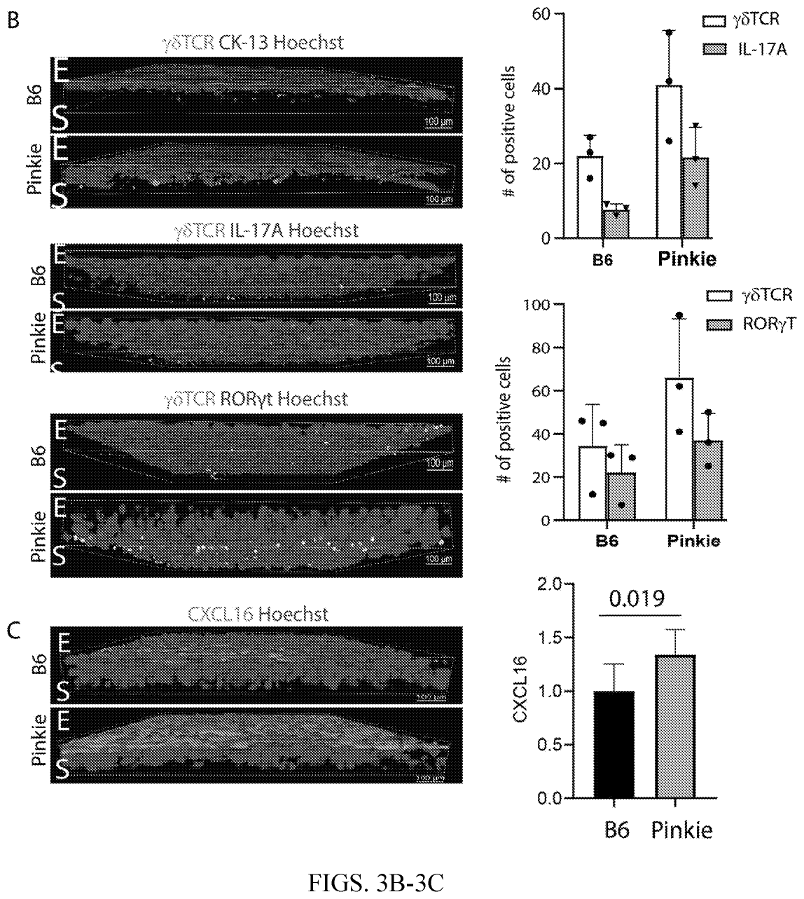

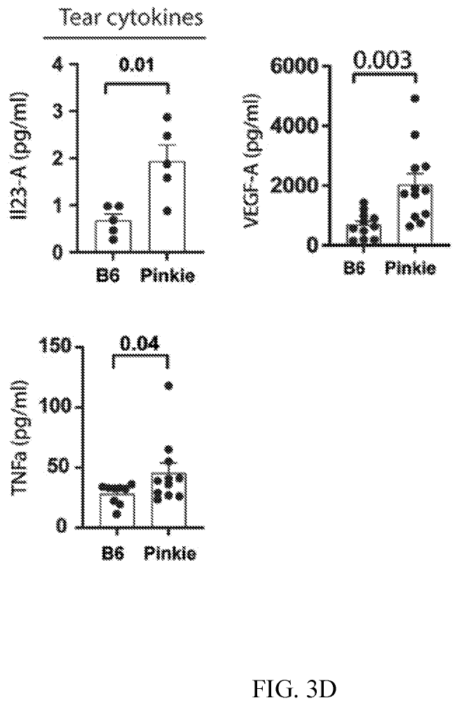

Flow cytometry shows an increased percentage of γδT cell receptor (TCR) negative and positive cells CD3⁺T cells and IL-17a⁺γδTCR⁻ and γδTCR⁺ cells in the Pinkie conjunctiva (FIG. 3A). 18 T cells are the predominant IL-17⁺ cells in both strains. Immunostaining of whole mount conjunctivas shows an increased number of total, IL-17a⁺ and RORγt⁺ γδ TCR⁺ cells in the conjunctiva (FIG. 3B). Minimal immunostaining for chemokine CXCL16, the ligand for CXCR6 that is expressed by γδT17 cells (Butcher et al., 2016) is noted in the B6 corneal epithelium, but strong staining is seen in the Pinkie conjunctival epithelium and is accompanied by increased mRNA expression in the conjunctival epithelium (FIG. 3C).

Increased concentrations of the γδT17 inducers IL-23 (Möhn et al., 2020) and TNF-α, (Lahn et al., 1998; Wu et al., 2014) as well as VEGF, a proangiogenic cytokine that promotes corneal neovascularization (Suryawanshi et al., 2012) (Li et al., 2011) are found in Pinkie tears (FIG. 3D).

### Example 5—9-cisRA Suppresses γδT17 Activation and Production of IL-23 by Monocytes

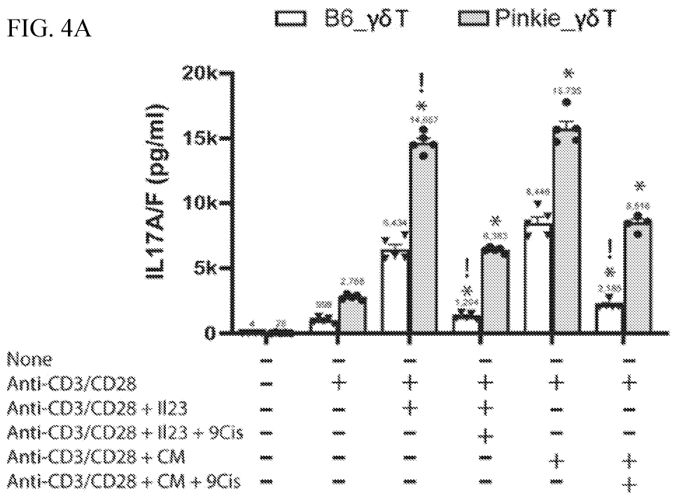

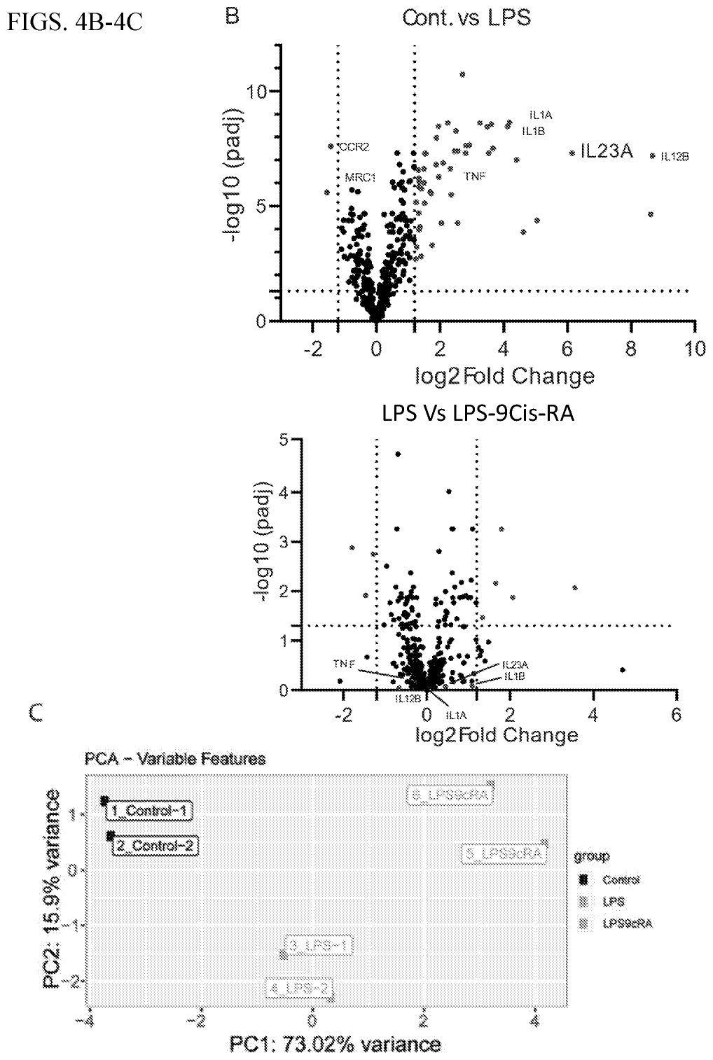

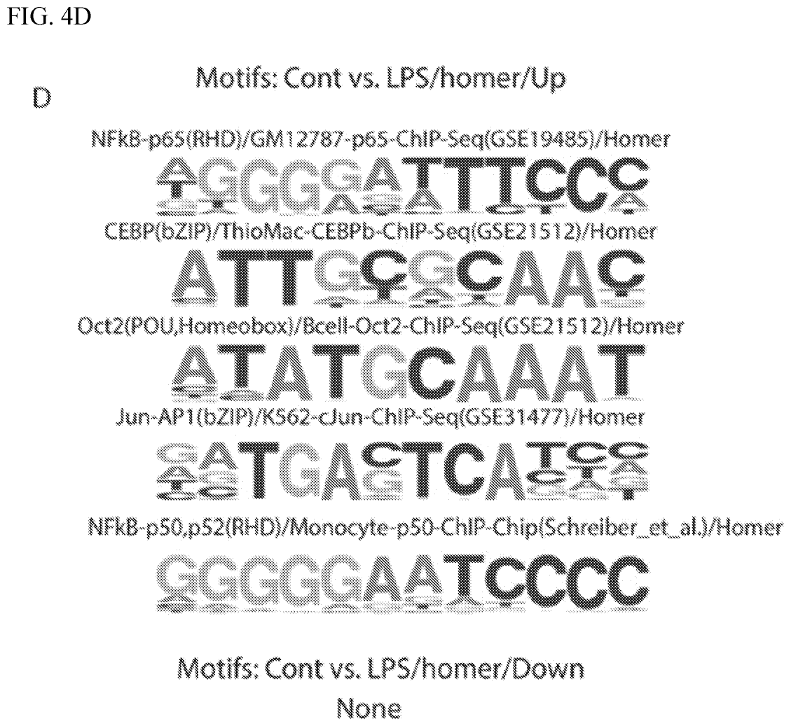

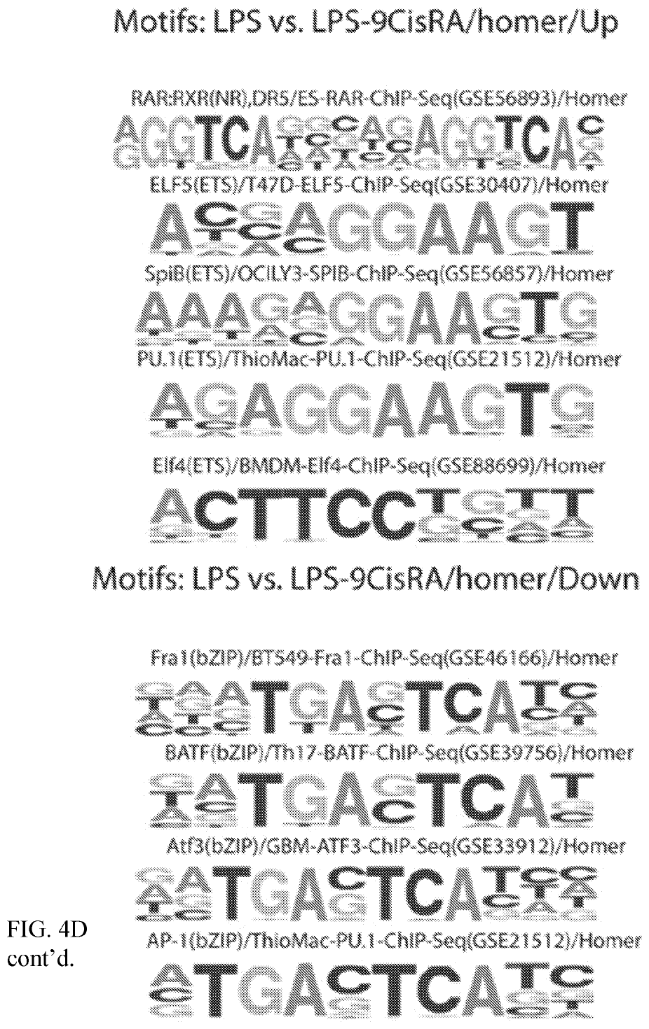

Based on the finding of increased γδT17 in the Pinkie conjunctiva, 9-cisRA was evaluated to determine if it suppresses IL-17 production by activated γδT cells in culture. γδT cells isolated from the spleen were stimulated with anti-CD3/CD28 beads with or without IL-23 and/or 9-cis RA. IL-17A/F was measured in the supernatant by ELISA. IL-17 release was higher in Pinkie γδT cells stimulated with beads or beads+IL-12 (FIG. 4A). 9-cis RA significantly reduced the supernatant IL-17 concentration in cells from both strains, although the suppressive effect was greater in the B6 cells (74% vs 46% in bead+IL-23 stimulated cells). The majority of myeloid cells in the conjunctiva express RXRα and when stimulated with LPS they produce factors known to stimulate IL-17 production by γδ T cells. (Alam et al., 2021b) The stimulatory activity of conditioned media from LPS-treated monocytes was compared to recombinant IL-23 on IL-17 production by γδ T cells and found they are equivalent (FIG. 4A). Furthermore, treatment of LPS-stimulated cultured monocytes with 9-cisRA significantly reduced stimulatory activity of their conditioned media (FIG. 4). Consistent with these findings, it was found that both genes encoding the IL-23 heterodimer (Il23a and Il12b), as well as other γδT17 inducing cytokines Il1α, Il1β and Tnf-α are significantly upregulated in LPS-stimulated cultured monocytes measured in a Nanostring array (FIG. 4B, top), and these are suppressed by addition of 9-cis RA to the culture media (FIG. 4B bottom).

Retinoic acid is known to cause epigenetic changes that can affect transcription factor binding and gene transcription. (Bar-El Dadon and Reifen, 2017) ATAC seq was performed on cultured monocytes to determine if 9-cis RA treatment changes the number of open transcription factor (TF) binding motifs in LPs-stimulated cultured monocytes. The PCA plot in FIG. 4C shows marked differences in peak region sequences in areas of open chromatin between control, LPS-treated and LPS+9-cis RA treated cells. LPS treatment significantly increased the number of TF motifs regulating transcription of inflammatory cytokines, including NFkB and Jun-AP-1 (FIG. 4D, top left), but didn't reduce the number of any known motifs (FIG. 4D, bottom left). Compared to LPS treatment alone, 9-cis RA+LPS increased the number of 5 known motifs, including RAR:RXR FIG. 4D (top right), and decreased the number of 4 motifs, including AP-1 FIG. 4D (bottom right). AP-1 is a key transcription factor for Il23a and other inflammatory mediators. (Liu et al., 2009)

Taken together, these findings indicate that RXRα suppresses production of IL-17 by activated γδ T cells and production of monocyte cytokines known to stimulate IL-17 production by γδT17 cells.

### Example 6—Differential Pathway Analysis Reveals Increased IL-17 Signaling in Pinkie

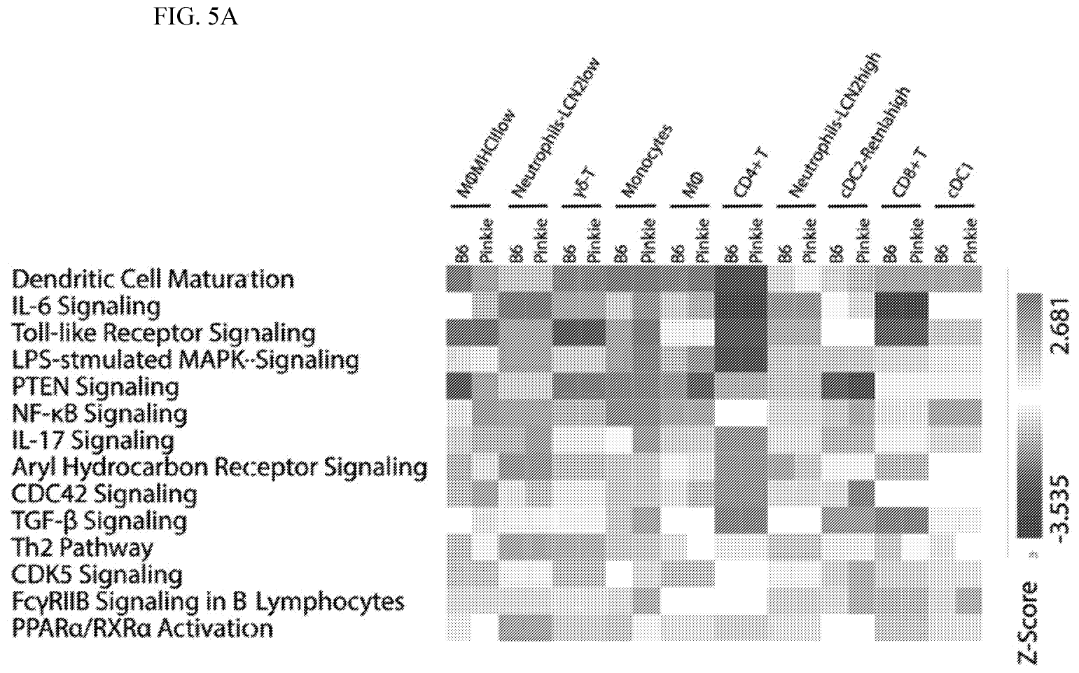

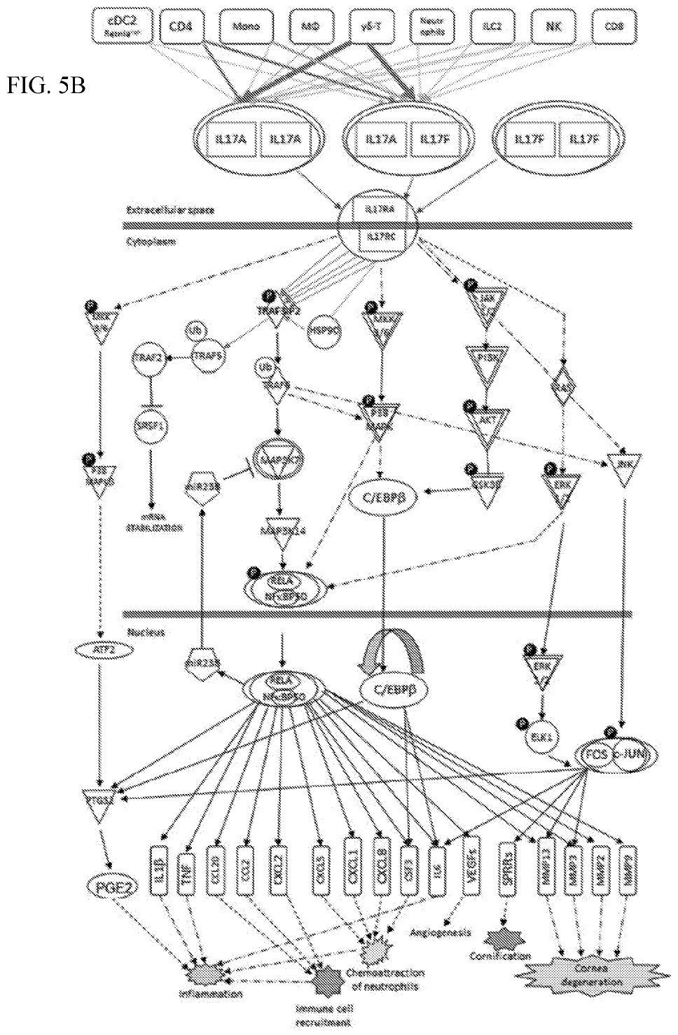

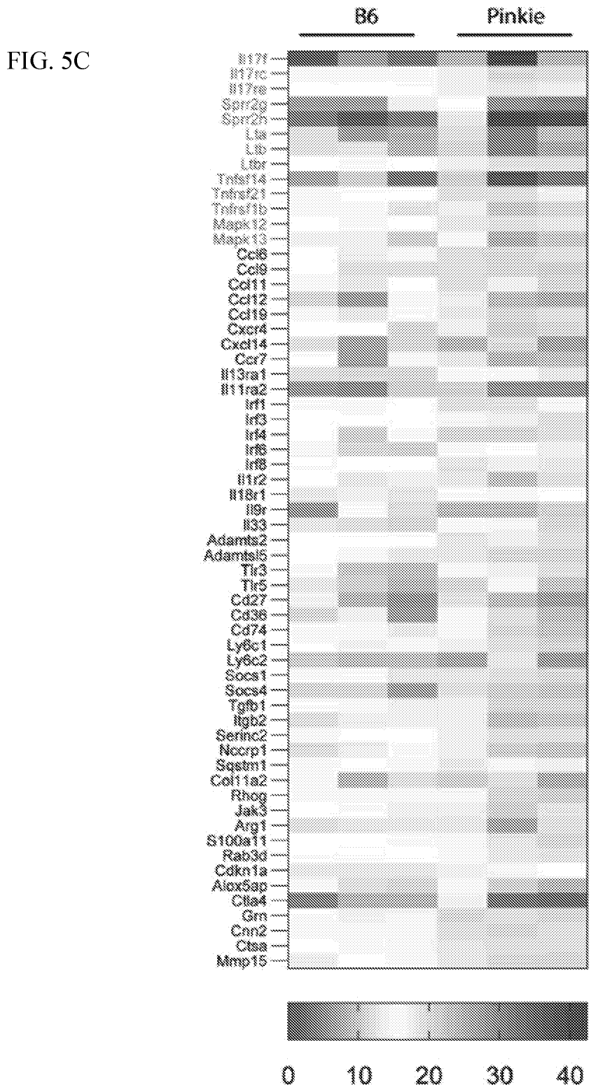

RXRα nuclear receptor regulates expression of an array of inflammatory mediators. QIAGEN Ingenuity Pathway Analysis (IPA) tool was used to identify significant differences (p≤0.05) between B6 and Pinkie in inflammatory signaling pathways generated from the scRNAseq data. These pathways grouped by strain and cell type are displayed in the heatmap shown in FIG. 5A. The greatest differences are seen in neutrophil, myeloid (macrophage and monocyte) and cDC2 cells and include IL-6, LPS-stimulated MAPK, NFkB, IL-17, and PPARα/RXRα signaling pathways that contain mediators relevant to dry eye pathogenesis. PPARα/RXRα signaling was significantly reduced in MHCII low macrophages and monocytes. Two other pathways, CDC42 and CDk5, have been implicated in NLRP3 inflammasome activation. (Müller et al., 2010; Cheng et al., 2020) The annotated IL-17 signaling pathway generated with IPA (FIG. 5B) contains downstream signaling pathways (MAPK and NFkB) that stimulate expression of γδT17 inducing cytokines and IL-17 inducible mediators (e.g. matrix metalloproteases, SPRR2) that are involved in development of the cornea and conjunctival epithelial disease of dry eye.

The conjunctiva is a mucosal tissue composed of epithelial, stromal and immune cells that express IL-17 receptors and are potential IL-17 targets. (McGeachy et al., 2019) To determine if IL-17 related genes/pathways are increased in the whole conjunctiva in Pinkie, expression profiles generated from bulk RNA seq performed on whole conjunctival lysates harvested from B6 and Pinkie were compared. Similar to the scSeq performed on immune cells, IL-17f together with IL-17 receptors (IL-17rc, IL-17re) was found to be among of the top differentially expressed genes with increased expression in Pinkie (FIG. 4C). There is also increased expression of other IL-17 signaling pathway associated genes, including cornified envelope precursor genes Sprr2g and Sprr2h (Lambert et al., 2017; Tian et al., 2021), p38 Mapks [Mapk12 (p38 gamma) and Mapk13 (P38 delta)], and chemokine CCL6. A variety of other inflammatory mediators and signaling molecules (e.g., Tlr3, Tlr5) are also increased. Taken together, this data indicates that RXRα suppresses production of IL-17 by γδ T cells and that IL-17 can exert protean influence on epithelial and immune cells on the ocular surface.

### Example 7—IL-17 Neutralization Suppresses Development of Ocular Surface Disease in Pinkie Bone Marrow Chimeras Exposed to Desiccating Stress

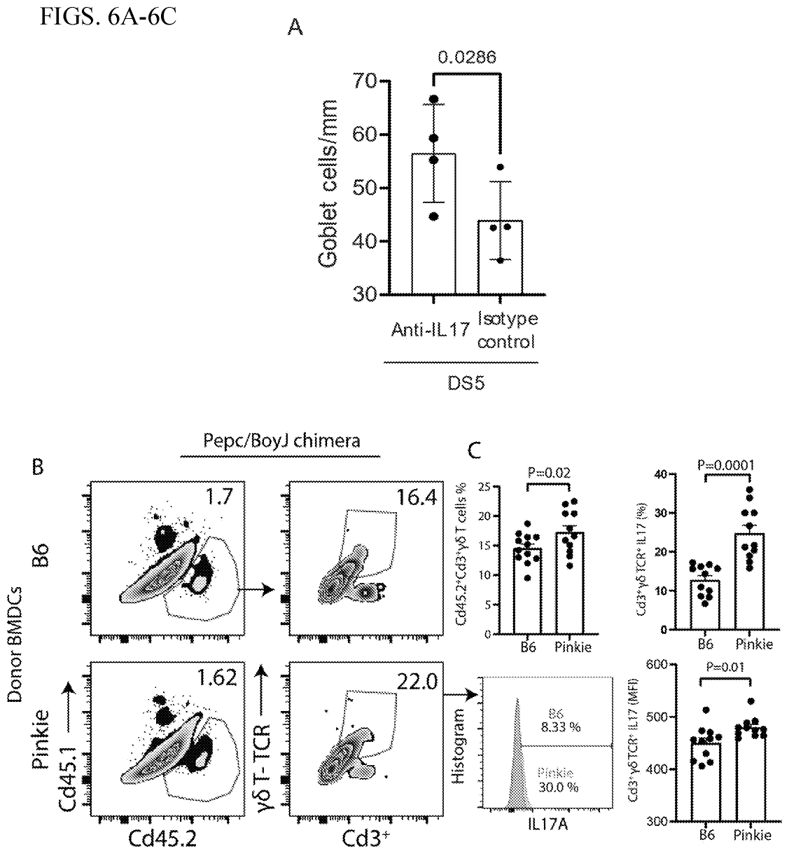

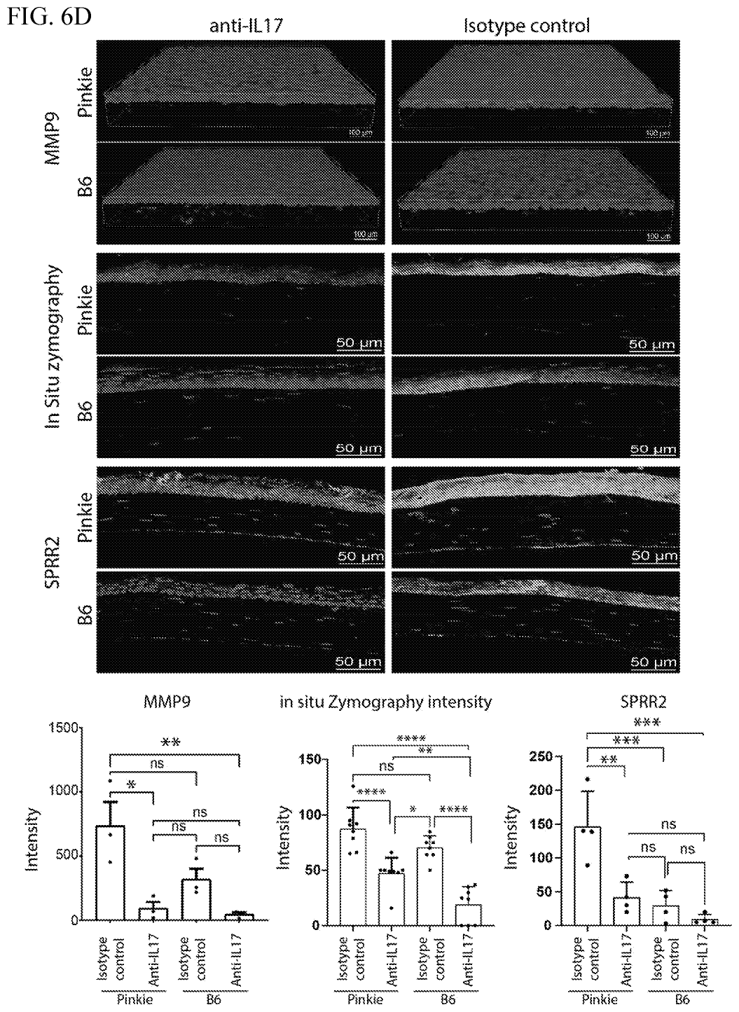

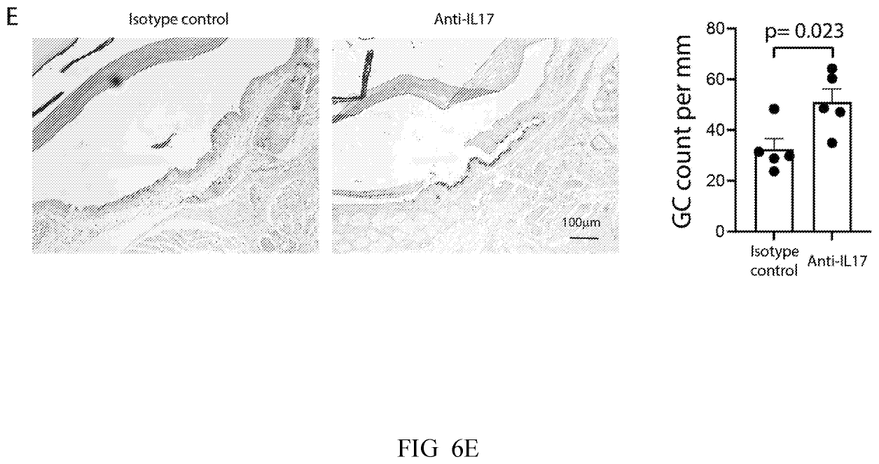

It was previously reported that IL-17 causes corneal barrier disruption in mice subjected to experimental desiccating stress (DS) by stimulating expression of metalloproteinases, (MMP-3 and MMP-9) that lyse tight junction proteins in the apical corneal epithelium (De Paiva et al., 2009). In that study, mice treated with anti-IL-17 had significantly less barrier disruption and reduced MMP-9 expression, MMP-9 immunostaining and gelatinase activity. In a previously unpublished experiment, it was also found that IL-17 neutralization prevented DS-induced conjunctival goblet loss (FIG. 6A).

Bone marrow chimeras created with Pinkie donor cells may produce greater ocular surface disease than those created with B6 donor cells because reduced RXRα signaling in Pinkie will lead to an increased infiltration of the conjunctiva by donor γδT17 cells. Bone chimeras created by a previously reported method (Alam et al., 2021a) and summarized in FIG. 11 were exposed to DS for 5 days. (Alam et al., 2021a) Chimeric mice were treated with either anti-IL-17 or isotype control antibodies every 2 days starting 2 days prior to initiating DS. After 5 days of DS, the percentages of γδT and IL-17+ cells in the conjunctiva were evaluated by flow cytometry and measures of dry eye disease, including corneal MMP-9 immunoreactivity and gelatinase activity (in situ zymography), and conjunctival goblet cell number. Pinkie donor chimeras were found to have a greater percentage of γδT cells and greater percentage and MFI of IL-17⁺γδTcells. MMP-9 immunoreactivity is significantly lower in anti-IL-17 treated than control treated Pinkie chimeras, and in situ gelatinase activity was lower in the anti-IL17 treated Pinkie and B6 chimeras (FIG. 6C). Anti-IL-17 treatment also reduced MMP-9 and SPPR2 immunostaining in the Pinkie corneal epithelium and gelatinase activity in the corneal epithelium of both strains (FIG. 6C). Conjunctival goblet cell density was significantly higher in the anti-IL-17 treated chimeras (FIG. 6E)

Taken together these data show that reduced RXRα signaling enhances migration of γδT17 cells to the conjunctiva in dry eye and that IL-17 produced by these cells causes corneal and conjunctival epithelial disease.

### Example 8—Pinkie Develops Corneal Neovascularization, Opacification and Ulceration with Aging

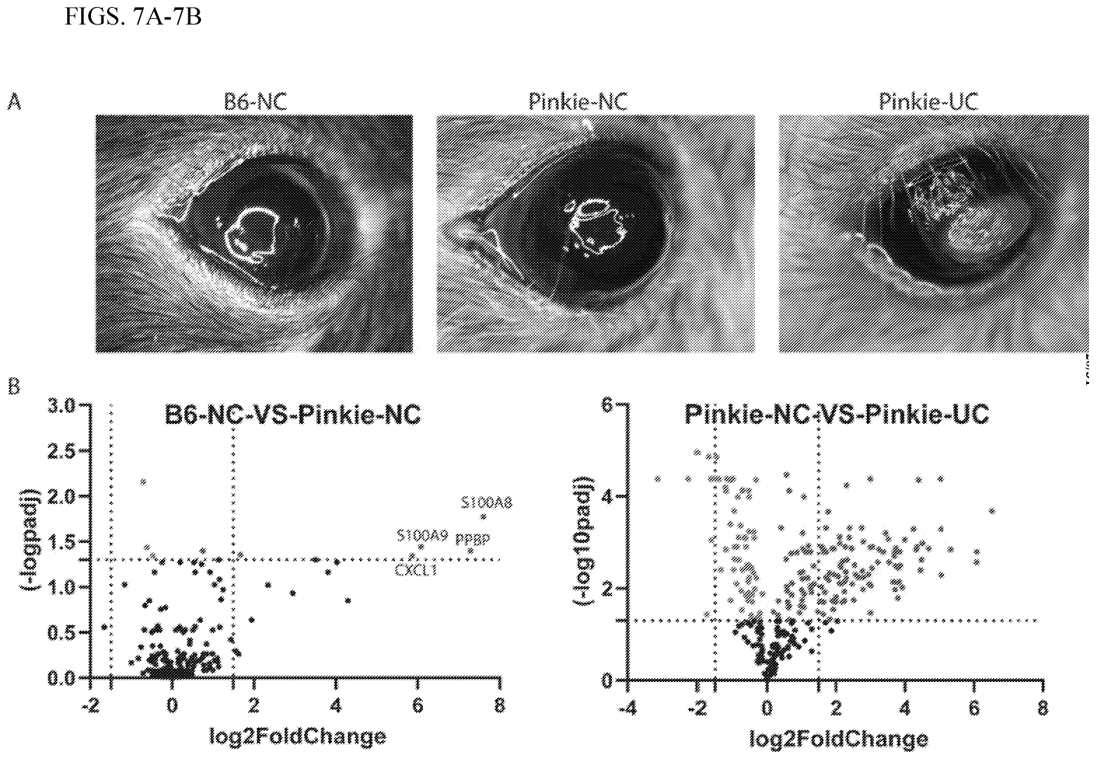

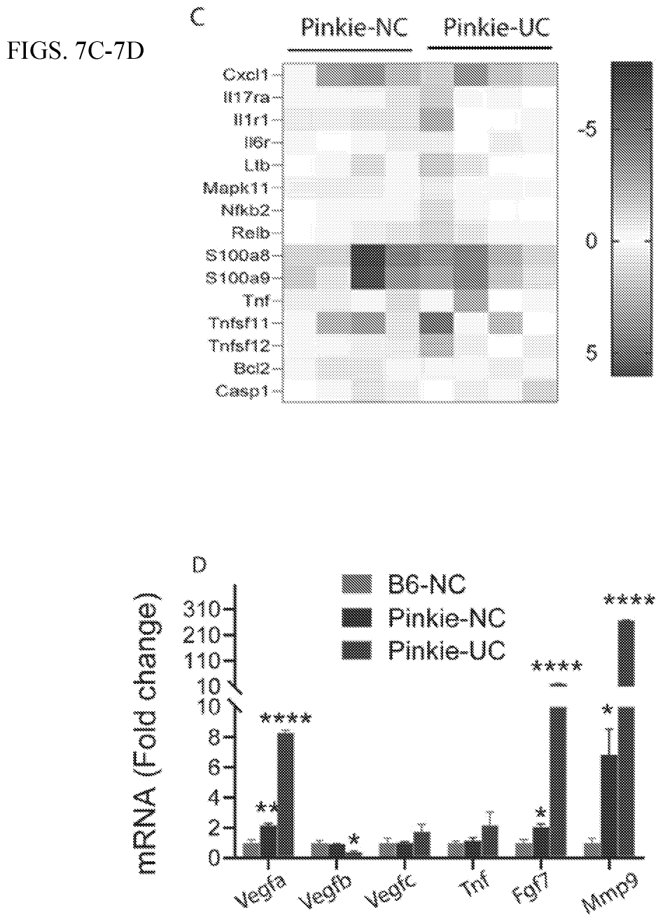

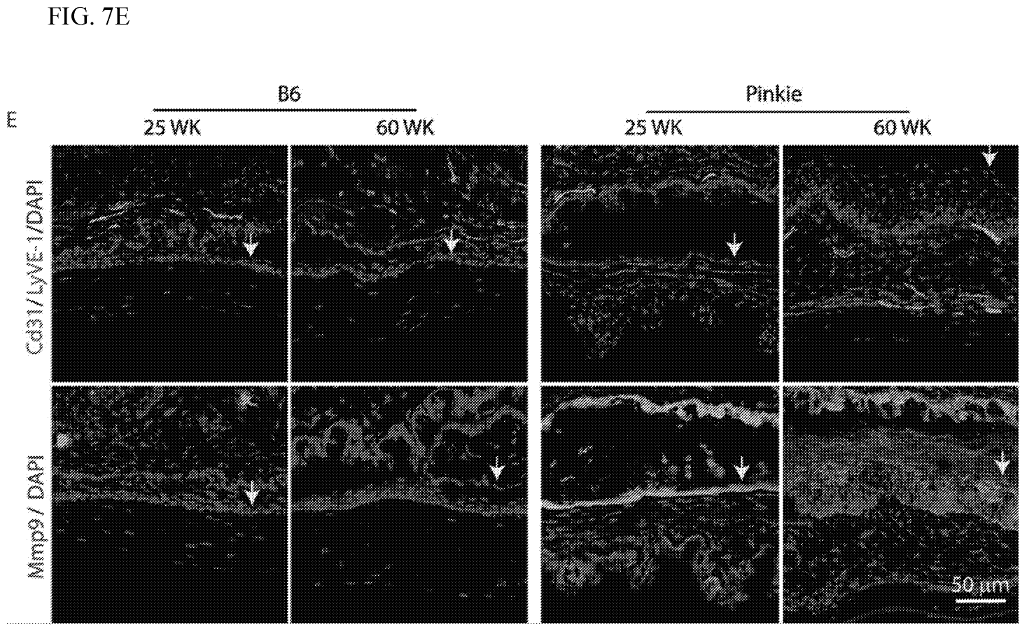

The Pinkie strain develops corneal opacification, neovascularization and ulceration with aging (FIG. 7A). Corneal opacity and vascularization was noted in 14% of 144 Pinkie eyes compared to only 2% of 100 B6 eyes. Gene expression profiles in Nanostring myeloid innate immunity arrays performed on whole cornea lysates prepared from 45-60 week old B6 or Pinkie with normal appearing corneas (NC) or from Pinkie with ulcerated corneas (UC) were compared. A violin plot shows 4 genes with significantly elevated expression in Pinke NC compared to B6 NC (FIG. 7B, top left). Significantly increased expression of numerous genes are noted when comparing normal and ulcerated Pinkie corneas (FIG. 7B, right). Included among the significantly differentially expressed genes in the Pinkie ulcerated cornea are the IL-17 signaling pathway genes displayed in the heatmap (FIG. 7C). These include γδT17 inducers (i.e., Ltb, Tnf, Nfkb2, Relb) and Il17ra.

Expression levels of several factors that promote corneal vascularization (Vegfa, Fgf7) and ulceration (Mmp9) measured by PCR are significantly increased in the Pinkie UC (FIG. 7D). Interestingly, expression of Vegfb, which has trophic activity on corneal nerves, was reduced in the Pinkie UC. (Guaiquil et al., 2014) Consistent with these findings is increased immunoreactivity of blood and lymphatic endothelial markers CD31/LYVE-1 and MMP-9 in the corneal epithelium of older Pinkie compared to similarly aged B6 (FIG. 7E). These findings suggest that dry eye combined with chronic elevation of pro-angiogenic and proteolytic factors in Pinkie promotes corneal vascularization, opacification and ulceration.

### Example 9—Mechanisms of RXRα in Dry Eye Disease

This study investigated the mechanism for developing dry eye disease in the Pinkie strain with a loss of function RXRα gene mutation. Using scRNA-seq as an unbiased approach to investigate the conjunctival immune cell population, a four-fold greater percentage of conjunctival γδ T cells that have higher expression of IL-17f and other γδ T17 signature genes were discovered. The sequencing findings are confirmed by flow cytometry and confocal microscopy that shows these cells are located in the stroma beneath the conjunctival epithelium. The Pinkie strain developed accelerated signs of dry eye disease in the cornea and conjunctiva. To determine the pathogenicity of Pinkie γδ T17 cells, bone marrow chimeras were created using Pinkie donor cells and found a significant reduction in corneal and conjunctival disease in the group receiving IL-17 neutralizing antibody.

IL-17 is involved in the pathogenesis of the corneal epithelial disease of dry eye. IL-17 stimulates MMP expression by the corneal epithelium, as well as neutrophil recruitment and activation. (De Paiva et al., 2009; Marzano et al., 2019) MMP-9 disrupts the corneal epithelial barrier via lysis of tight junction proteins in the apical epithelium that results in accelerated desquamation. (Pflugfelder et al., 2005) Conjunctival goblet cell loss in dry eye can develop from cytokine mediated apoptosis or altered differentiation with entrapment of goblet cells by abnormally differentiated epithelium with increased expression of cornified envelope precursors such as SPRR2 which is induced by IL-17.

Previously reported studies found antibody neutralization of IL-17 significantly reduces corneal barrier disruption measured by OGD permeability in the desiccating stress model of dry eye. While performing those studies, it was also found that anti-IL-17 prevented desiccation induced conjunctival goblet cell loss. Studies reported by others have also found that IL-17 produced by Th17 cells causes cornea and conjunctival disease. (Chen J Immunol; Chauhan J Immunol 2009). IL-17 is primarily produced by CD4⁺ T cells and γδ T cells. IL-17 was detected in CD4⁺ T cells by flow cytometry in previous studies using the DS dry eye model, but most didn't evaluate IL-17 production by conjunctival γδ T cells. Increased expression of IL-17 was noted in conjunctival epithelium of patients with Sjogren syndrome keratoconjunctivitis sicca, but the cellular source was not determined. (Pflugfelder et al., 2015) γδ T cells were the second most prevalent population of intraepithelial lymphocytes in the mouse conjunctiva (Zhang et al., 2012), and Coursey et al. reported IL-17 is produced by γδ T cells in the lacrimal glands of the NOD mouse strain that develops KCS and is used as a model of SS. (Coursey et al., 2016) This study suggests that conjunctival γδ T cells are another source of IL-17 and that IL-17 expression in these cells is regulated by the RXRα nuclear receptor. γδT cells are found in many mucosal surfaces and can be activated in a non-antigen specific manner by a variety of PAMPs and conceivably to desiccating stress that activates the same signaling pathways as microbial products. (Hedges et al., 2005)

The RXR nuclear receptor family regulates transcription of numerous genes involved in immune function, cell differentiation and homeostasis. RXRα may function as a homodimer or a heterodimer with partner receptors (PPARγ and the vitamin D receptor) that have been found on the ocular surface. (Nien et al., 2010; Panigrahi et al., 2021) The ocular surface is a retinoid rich environment. (Alam et al., 2021b) Besides the retinol form of vitamin A in tears that is converted to the natural ligand 9-cis RA by aldehyde dehydrogenases in myeloid and epithelial cells on the ocular surface (Xiao et al., 2018), nutritional ligands such as vitamin D, the omega-3 fatty acid DHA in fish oil and oleic acid in olive oil can bind certain RXR dimeric partners. (Alam et al., 2020a)

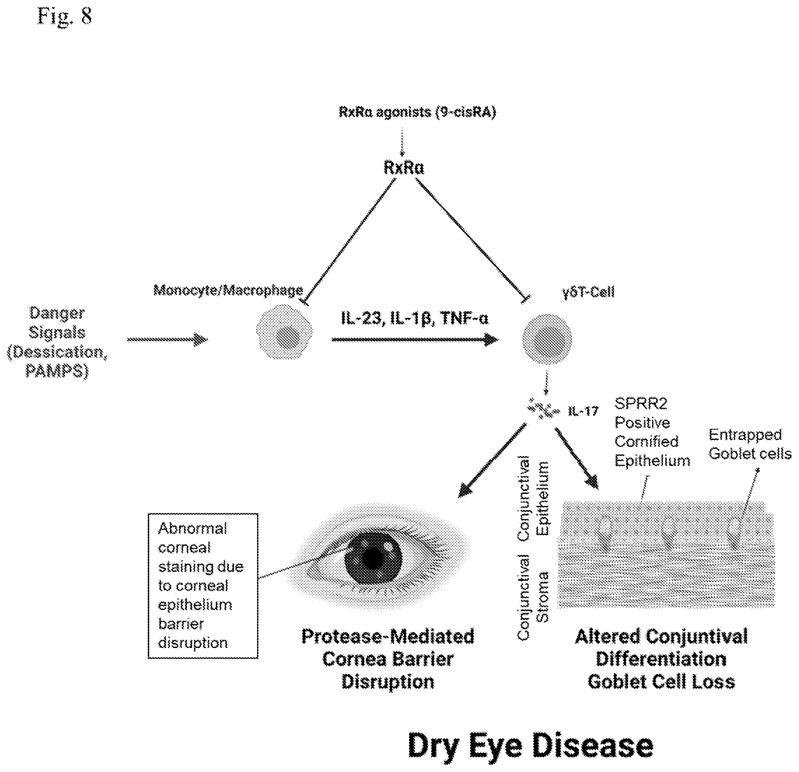

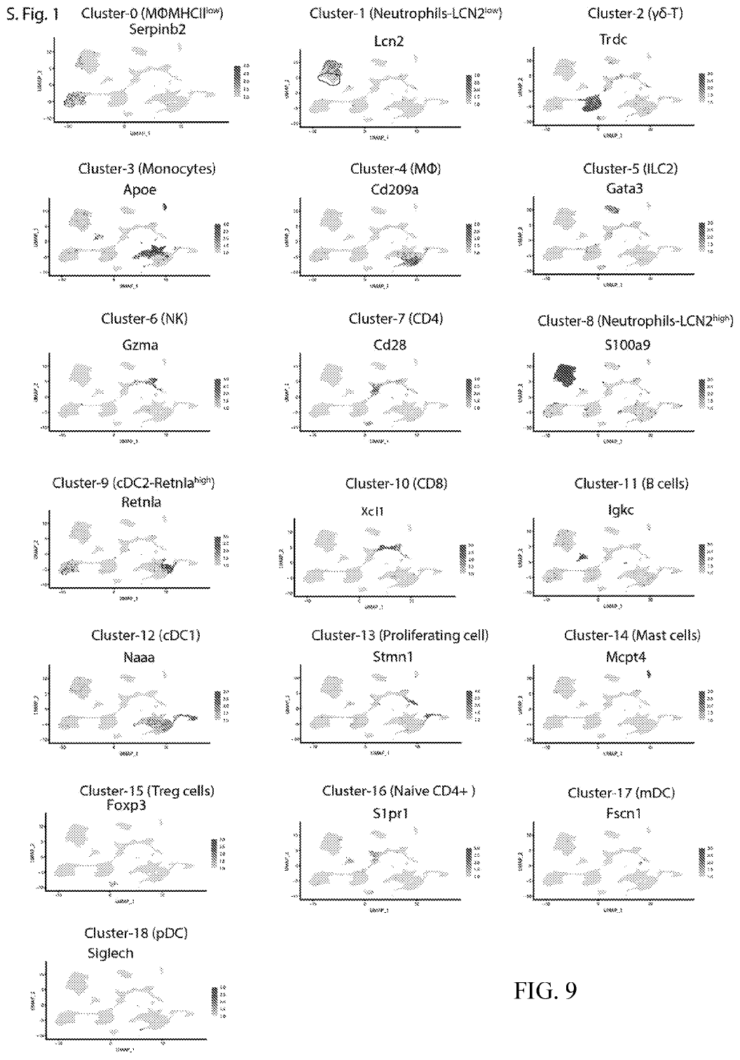

The majority of CD11b+ myeloid cells are RXRα positive and respond to retinoic acid. (Alam et al., 2021b) The discovery of increased IL-17 producing γδ T cells in the Pinkie strain indicates RXRα is also an important regulator of IL-17 production by γδ T cells. The synthetic retinoid AM80 was found to suppress IL-17 production by γδ T cells stimulated with anti-CD28 antibody and a cytokine cocktail of IL-23 and IL-1. (Möhn et al., 2020) 9-cisRA suppresses IL-17 production by >70% in cultured γδ T cells stimulated by CD28 beads or beads plus IL-23. In addition to direct suppression of γδ T cells, was also found 9-cisRA suppresses expression of γδ T17 inducers (IL-23, IL-1, TNF-α) by cultured monocytes and it was previously reported that reduced levels of IL-1β and IL-23β in supernatants of 9-cis RA treated monocytes. (Alam et al., 2021b) Monocyte conditioned media has stimulatory activity equivalent to recombinant IL-23, but this was significantly reduced in monocytes cultured with 9-cis RA. It was also found that 9-cis RA decreases the number of open AP-1 transcription factor binding motifs detected by ATAC seq. Both AP-1 and NFkB pathways are involved in stimulated IL-17 expression by γδ T cells. (Powolny-Budnicka et al., 2011; Criado et al., 2014) FIG. 8 summarizes the primary and secondary suppressive activity of 9-cis RA on production of IL-17 by γδ T cells.

Single cell profiling was performed on conjunctival immune cells because it is difficult to obtain a sufficient number of donor cells from the cornea. It is possible the corneal pathology results from IL-17 produced by conjunctival γδ T cells, but IL-17 producing γδ T cells have been found to infiltrate the cornea following epithelial trauma. (Li et al., 2007; Li et al., 2011) ATAC seq was performed on monocytes, which demonstrated epigenetic effects of 9-cis RA has on these cells. The discovery that RXRα suppresses IL-17 production by γδ T cells is rationale for evaluating epigenetic activity of 9-cis RA on these cells in the future.

The findings indicate that RXRα retinoid signaling suppresses activation and IL-17 production by moused conjunctival γδ T cells under homeostatic conditions. This signaling may be reduced in aqueous tear deficient dry eye due to reduced secretion of retinol into tears by dysfunctional lacrimal glands, in some embodiments. Additionally, there could be decreased aldhehyde dehydrogenase expression in the conjunctiva in dry eye that could result in decreased RA synthesis, in some embodiments. Strategies that maintain the ocular surface retinoid axis in dry eye may prevent IL-17 induced epithelial pathology, in some embodiments.

TABLE 1
Cluster Identity

| | | | | | | CIPR Cluster Identification | | |
|---|---|---|---|---|---|---|---|---|
| | | C57BL_6 | | Pinkie | | reference | | percent_pos_{—} |
| Cluster | Name | Count (%) | | Count (%) | | cell_type | reference_id | correlation |
| 0 | MΦMHCII^{low} | 3239 | (29.0) | 181 | (1.6) | Macrophage | MF.II-480hi.PC | 60 |
| 1 | Neutrophils- | 2252 | (20.2) | 750 | (6.7) | Granulocyte | GN.Thio.PC | 100 |
| | LCN2^{low} | | | | | | | |
| 2 | γδ-T | 434 | (3.9) | 1820 | (16.3) | γδ-T cell | Tgd.vg2 + 24alo.Th | 93.33 |
| 3 | Monocytes | 1180 | (10.6) | 782 | (7.0) | Macrophage | MF.11c-11b + .Lu | 90.47 |
| 4 | MΦ | 889 | (8.0) | 402 | (3.6) | DC | DC.103-11b + 24 + .Lu | 100 |
| 5 | ILC2 | 349 | (3.1) | 492 | (4.4) | ILC-2 | ILC2.SI | 100 |
| 6 | NK cells | 481 | (4.3) | 291 | (2.6) | NK cell | NK.CD127 − .SI | 100 |
| 7 | CD4⁺ T cells | 249 | (2.2) | 484 | (4.3) | T cell | T.4Mem49d + 11a + | 100 |
| | | | | | | | .Sp.d30.LCMV | |
| 8 | Neutrophils- | 454 | (4.1) | 247 | (2.2) | Granulocyte | GN.Thio.PC | 100 |
| | LCN2^{high} | | | | | | | |
| 9 | cDC2- | 460 | (4.1) | 209 | (1.9) | DC | DC.11b + .AT.v2 | 100 |
| | Retnla^{high} | | | | | | | |
| 10 | CD8⁺ T cells | 209 | (1.9) | 301 | (2.7) | NK cell | NK.CD127 − .SI | 100 |
| 11 | B cells | 121 | (1.1) | 275 | (2.5) | B cell | B.T2.Sp | 100 |
| 12 | cDC1 | 212 | (1.9) | 168 | (1.5) | DC | DC.8 − . Th | 100 |
| 13 | Proliferating | 148 | (1.3) | 185 | (1.7) | Pre-T cell | T.DPbl.Th | 92.30 |
| | cell | | | | | | | |
| 14 | Mast cells | 202 | (1.8) | 113 | (1.0) | Mast cell | MC.Tr | 98.24 |
| 15 | Treg cells | 113 | (1.0) | 186 | (1.7) | Treg | ABD.TR.14w.B6 | 100 |
| 16 | Naive CD4+ | 83 | (0.7) | 123 | (1.1) | T cell | CD4.1h.LN | 100 |
| 17 | Migratory DC | 62 | (0.6) | 63 | (0.6) | DC | DC.IIhilang-103- | 100 |
| | (mDC) | | | | | | 11b + .SLN | |
| 18 | Plasma- | 28 | (0.3) | 21 | (0.2) | DC | DC.pDC.8 − .Sp | 100 |
| | cytoid DC | | | | | | | |
| | (pDC) | | | | | | | |

| | |
|---|---|
| 11165 | 7093 |

TABLE 2
Antibodies
Antibodies in this study

| Antibody | | | | | | Company | |
|---|---|---|---|---|---|---|---|
| Type | Target | Clone | Host | Reaction | Conjugation | (Catalog No.) | Dilution |
| Primary | CXCL16 | 142417 | Rat | Mouse | None | ThermoFisher | 1:40 |
| | | | | | | (MA5-23869) | |
| Primary | γδ T-Cell | GL3 | Ham | Mouse | FITC | BD | 1:30 |
| | Receptor | | | | | (553177) | |
| Primary | RORγt | Q31-378 | Mouse | Mouse | PE | BD | 1:30 |
| | | | | | | (562607) | |
| Primary | IL-17A | TC11- | Rat | Mouse | PE | BD | 1:30 |
| | | 18H10 | | | | (559502) | |
| Primary | Cyto- | CK-13 | Rabbit | Mouse | None | ABclonal (A0411) | 1:40 |
| | keratin 13 | | | | | | |
| | (KRT13) | | | | | | |
| Primary | SPRR2 | Polyclonal | Rabbit | Mouse | None | ABclonal (Custom- | 1:40 |
| | | | | | | made using | |
| | | | | | | 'KCPEPCPPPKCPQPC | |
| | | | | | | PP' peptide sequence) | |
| Secondary | IgG | Polyclonal | Goat | Rat | Alexa | Jackson | 1:1000 |
| | (H +L) | | | | Fluor 488 | ImmunoResearch | |
| | | | | | | (112-546-143) | |
| Secondary | IgG | Polyclonal | Goat | Rabbit | Alexa | Jackson | 1:1000 |
| | (H + L) | | | | Fluor 488 | ImmunoResearch | |
| | | | | | | (111-545-003) | |
| Secondary | IgG | Polyclonal | Goat | Rabbit | Alexa | Jackson | 1:1000 |
| | (H + L) | | | | Fluor 594 | ImmunoResearch | |
| | | | | | | (111-585-003) | |

Proteintech, Rosemont, IL;
Thermofisher Scientific, Waltham, MA;
Abcam, Waltham, MA Abclonal, Woburn, MA;
Becton Dickinson (BD), San Diego, CA;
R&D Systems, Minneapolis, MN Jackson ImmunoResearch, West Grove, PA;
Ham = hamster

TABLE 3

| Cluster | Name | Top 20 genes of each cluster |
|---|---|---|
| 0 | MΦMHCII^{low} | Serpinb2, F5, Alox15, Basp1, Gadd45a, Ccl6, Cd24a, Rflnb, |
| | | Ccl9, Tgm2, Irf2bp2, Cyp4f18, Vegfa, P2ry10, Arpc5, Plac8, |
| | | Kctd12, Dnajb6, Thbs1, Itgam |
| 1 | Neutrophils- | Il1r2, Ifitm1, Tnfaip2, Nlrp3, Marcksl1, Csf3r, Ccrl2, Trem1, |
| | LCN2^{low} | Clec4d, Clec4e, S100a8, Clec4n, S100a9, Il1b, Hdc, Resf1, Cd14, |
| | | Acod1, C5ar1, Lrg1 |
| 2 | γδ-T | Il17a, Trdc, Cxcr6, Areg, Cd163l1, Tcrg-C1, Icos, Cd3g, Rora, Emb |
| | | Cd3e, Trdv4, Blk, Ikzf3, Pdcd1, Il7r, Nrip1, Thy1, Cd3d, Tnfrsf9 |
| 3 | Monocytes | Apoe, Lyz2, Mafb, Lgmn, Selenop, Ms4a6c, C1qa, F13a1, Chil3, |
| | | Ctss, C1qb, Ctsc, Ifi207, Ifitm3, Ctsb, Mrc1, Ifi27l2a, Ccr2, Ly86, |
| | | C1qc, |
| 4 | MΦ | Cd209a, Tnip3, Cd74, H2-Ab1, H2-Eb1, H2-Aa, Gm26917, |
| | | Mgl2, Mt1, Cd83, Ifi30, Mt2, Etv3, Clec10a, H2-DMb1, H2- |
| | | DMa, Syngr2, Sdc4, Ccnd1, |
| | | Crip1 |
| 5 | ILC2 | Il5, Gata3, Areg, Csf2, Rora, Lmo4, Ccl1, Nrip1, Furin, Itk, Il13, |
| | | Il2ra, |
| | | Inpp4b, Tmem64, Calca, Ctla2a, Arg1, Hs3st1, Uhrf2, Il7r |
| 6 | NK cells | Gzma, Ccl5, AW112010, Nkg7, Irf8, Klra4, Il2rb, Prf1, Serpinb9, |
| | | Klra8, Eomes, Gzmb, Ugcg, Klre1, Serpinb6b, Sh2d2a, Spry2, |
| | | Ms4a4b, Txk, Klra9 |
| 7 | CD4⁺ T cells | Cd28, Icos, Trac, Ets1, Rora, S1pr1, Ctla4, Thy1, Uhrf2, Il18r1, |
| | | Trbc2, Ms4a4b, Ptpn22, Il7r, Trbc1, Emb, Tcf7, Lat, Ramp3, |
| | | Cd3d |
| 8 | Neutrophils- | S100a9, S100a8, Lcn2, Stfa2l1, Wfdc21, Slfn4, Cstdc4, Lrg1, Hp, |
| | LCN2^{high} | Clec4d, Slpi, Csf3r, Trem1, Acod1, Cxcr2, Hdc, Msrb1, Grina, Isg15, |
| | | Oasl2 |
| 9 | cDC2- | Lpl, Retnla, Fn1, Lyz1, Fcrls, Ear2, Mt1, Pltp, Mt2, Lyz2, Cd74, |
| | Retnla^{high} | Mgl2, H2-DMa, Crip1, H2-DMb1, H2-Ab1, Tnip3, Flrt3, Sdc3, H2-Eb1 |
| 10 | CD8⁺ T cells | Ccl5, Xcl1, Il2rb, Ms4a4b, Gimap4, Trbc2, Ctsw, Serpinb6b, |
| | | Klrc1, Nkg7, Gimap3, Ptpn22, Cd28, Ly6c2, AW112010, Klrk1, |
| | | Ctla2a, Serpinb9, Txk, Ets1 |
| 11 | B cells | Igkc, Cd79a, Ebf1, Ighm, Ighd, Scd1, Ms4a1, Cd79b, Mef2c, |
| | | Ly6d, Pax5, H2-Ob, Satb1, Ccr7, Fcmr, Fcer2a, Ralgps2, Cd19, |
| | | Cd55, H2-DMb2 |
| 12 | cDC1 | Cst3, Naaa, Wdfy4, Ppt1, H2-Ab1, H2-Eb1, 44442, Rab7b, H2-Aa, |
| | | Plbd1, H2afz, Ifi205, Tmsb10, Cd74, Naga, Irf8, Mpeg1, |
| | | Trim35, Eef1b2, Atpif1 |
| 13 | Proliferating | Stmn1, Tubb5, Ighm, Top2a, Pclaf, Tuba1b, Mki67, Ube2c, H2afz, |
| | cell | Hmgb2, H2afv, Birc5, Hist1h2ap, Anp32b, Dek, Tmsb10, Hist1h1b, |
| | | Crip1, Nucks1, Hmgb1 |
| 14 | Mast cells | Mcpt4, Cpa3, Cma1, Tpsb2, Mrgprb1, Jun, Serpinb1a, Kit, Egr1, |
| | | Slc18a2, Gata2, Ccl2, Mrgprb2, Ube2v2, Cyp11a1, Tpsab1, |
| | | Vwa5a, Fcer1a, Ccl7, Hs6st2 |
| 15 | Treg cells | Ctla4, Tnfrsf4, Ikzf2, Il2ra, Icos, Hopx, Odc1, Cpm, Trac, Foxp3, |
| | | Tnfrsf9, Rora, Cd2, Tnfrsf18, Cd28, Maf, Trp53inp1, Gata3, Ets1, |
| | | Trbc2 |
| 16 | Naive CD4+ | Satb1, S1pr1, Dusp10, Lef1, Ccr7, Ms4a4b, Gimap3, Gramd3, Tcf7, |
| | | Trbc2, Gimap6, Ablim1, Rpl12, Bcl2, Il7r, Ets1, Txk, Ms4a6b, |
| | | Rps20, Trib2 |
| 17 | Migratory DC | Ccr7, Ccl22, Fscn1, Tbc1d4, Fabp5, Apol7c, Il4i1, Clic4, Cxcl16, |
| | (mDC) | Ccl17, Clu, Tmem123, Ifi30, Serpinb9, Tspan3, Nrp2, Socs2, Ccl5, |
| | | Cacnb3, Serpinb6b |
| 18 | Plasmacytoid | Siglech, Tcf4, Bst2, Ccr9, Ly6d, Klk1, Irf8, Ly6c2, Spib, Atp1b1, |
| | DC (pDC) | Lair1, Cox6a2, Lrp8, Ctsl, Cyb561a3, Bcl11a, Rpl31, Runx2, Grn, |
| | | Mpeg1 |

### Example 10—Examples of Effects of RXRα Agonists

The present example shows the suppressive effects of examples of RXRα agonists on IL-17 production by cultured γδ T cells, and differences in immune cells and gene expression between wild type C57BL/6 and Pinkie mice. Similar to the conjunctiva, there was an increase in IL-17 producing γδ T cells, which shows the importance of RXRα in suppressing this key inflammatory cytokine.

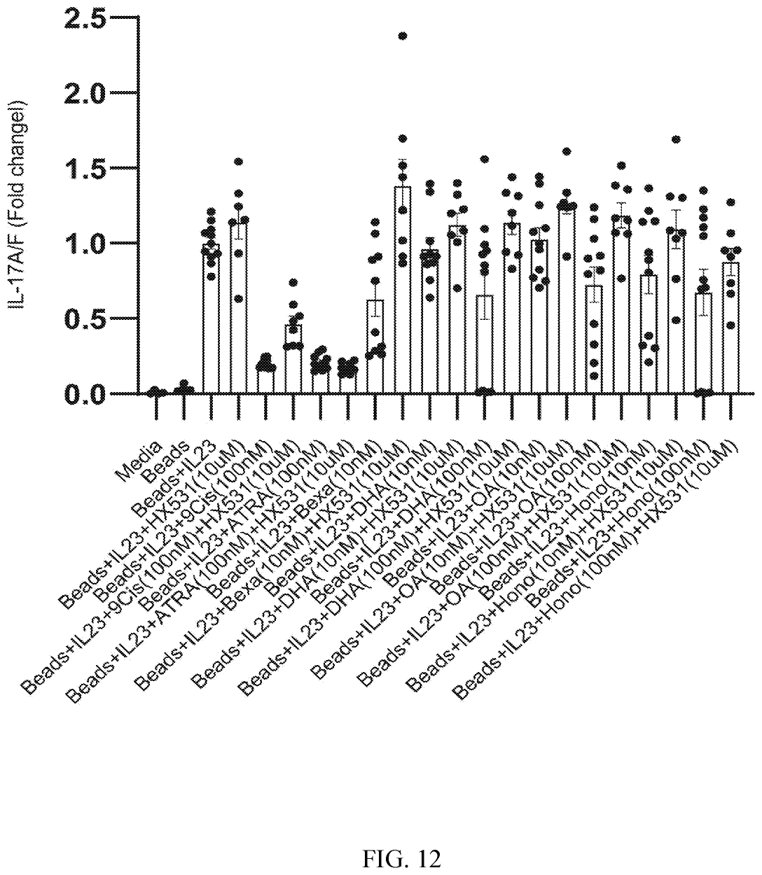

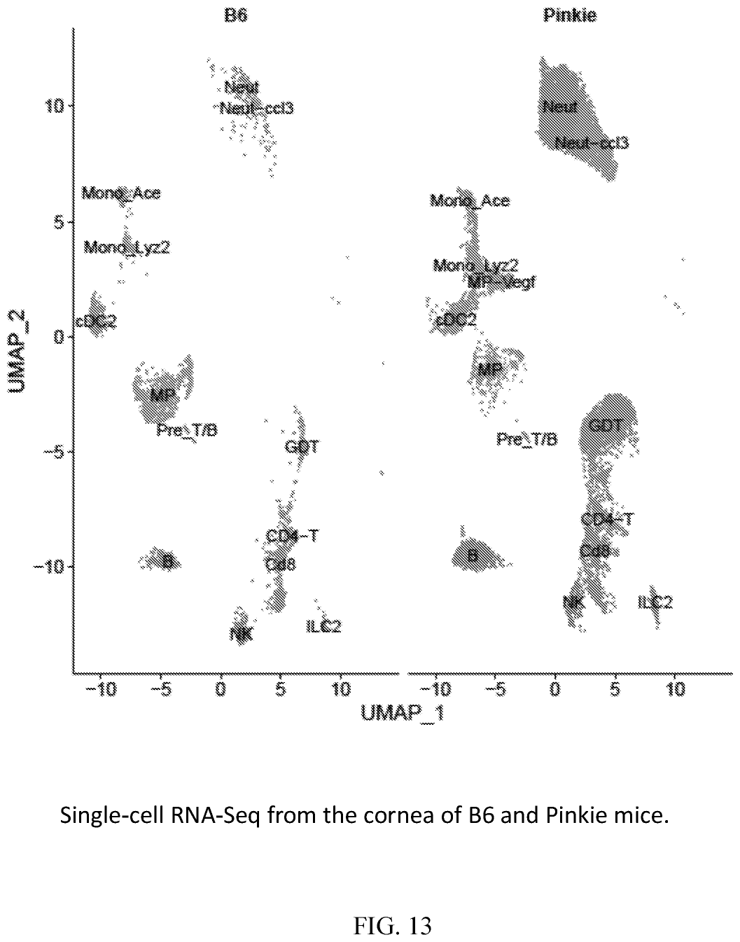

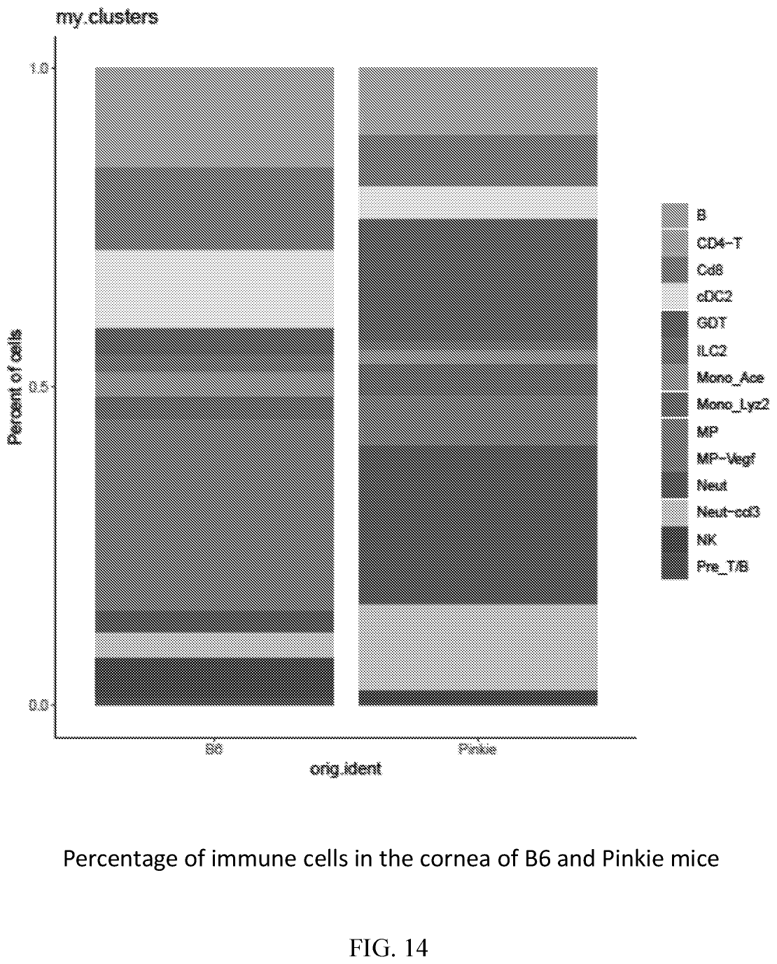

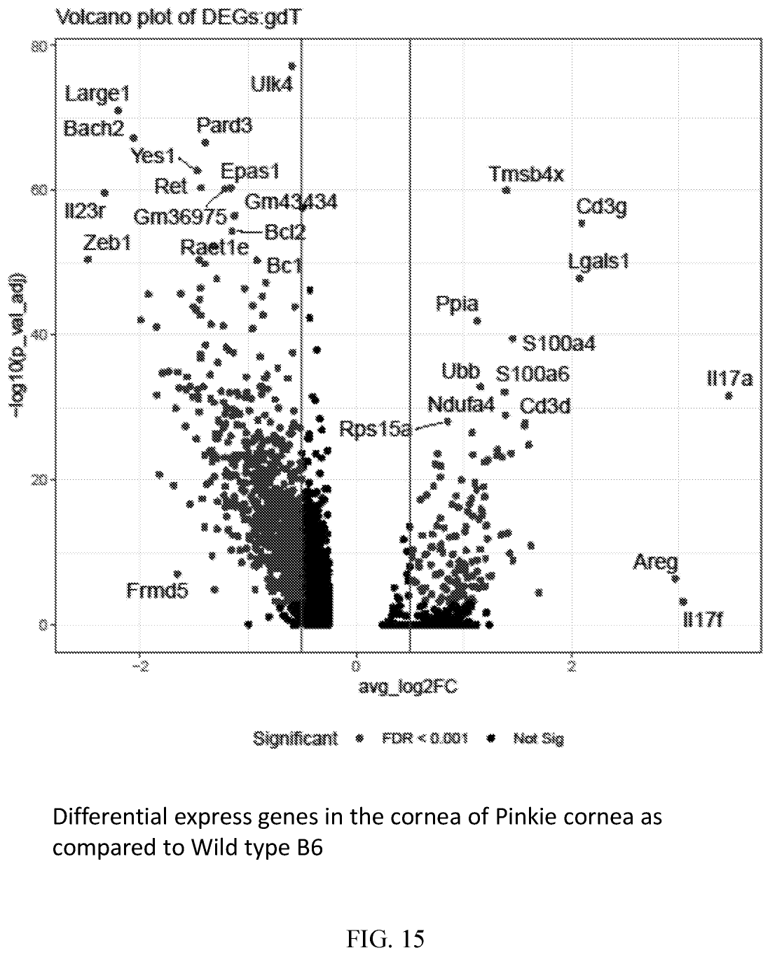

FIG. 12 shows IL-17A/F cytokine concentration measured in supernatants of cultured mouse γδ T cells isolated with magnetic beads from C57BL/6 spleens. Cells are stimulated with anti-CD3/CD28 beads plus recombinant IL-23 without or with addition of RXRα agonists 9-cisRA (9-Cis), Bexarotene (Bexa), omega 3 fatty acid DHA, fatty acid oleic acid (OA), and honokiol (Hono) at concentrations of 10 and 100 nM. RXRα antagonist HX531 blocked the suppressive effects of all agonists, but did not have any effect on suppressive activity of the RAR agonist all trans retinoic acid (ATRA). FIG. 13 provides UMAP of 14 distinct immune cell clusters in corneas from C57B/6 (B6) and Pinkie generated from single-cell transcriptomic profiles of CD45⁺ cells using Seurat package v3. FIG. 14 demonstrates heatmaps comparing the percentages of 14 cell types in B6 and Pinkie corneas. The greatest changes are an increase in γδ T cells and neutrophils and a decrease in macrophages (MP) in Pinkie corneas. FIG. 15 shows a volcano plot comparing level of differentially expressed genes in corneas of B6 and Pinkie strains. A mouse myeloid Innate Immunity Nanostring array was used to evaluate gene expression. Solid vertical lines indicate less or greater than 0.5 log 2 fold change. Red dots are significantly increased (right) or decreased (left) genes. IL-17a and IL-17f are among the significantly increased genes.

### REFERENCES

The following references, to the extent that they provide exemplary procedural or other details supplementary to those set forth herein, are specifically incorporated herein by reference.
Alam, J., De Paiva, C. S., and Pflugfelder, S. C. (2020a). Immune—Goblet Cell Interaction in the Conjunctiva. Ocul Surf.
Alam, J., De Paiva, C. S., and Pflugfelder, S. C. (2021a). Desiccation Induced Conjunctival Monocyte Recruitment and Activation—Implications for Keratoconjunctivitis. Front Immunol 12, 701415.
Alam, J., De Souza, R. G., Yu, Z., Stem, M. E., De Paiva, C. S., and Pflugfelder, S. C. (2020b). Calcineurin Inhibitor Voclosporin Preserves Corneal Barrier and Conjunctival Goblet Cells in Experimental Dry Eye. J Ocul Pharmacol Ther.
Alam, J., Yu, Z., De Paiva, C. S., and Pflugfelder, S. C. (2021b). Retinoid Regulation of Ocular Surface Innate Inflammation. Int J Mol Sci 22.
Bar-El Dadon, S., and Reifen, R. (2017). Vitamin A and the epigenome. Crit Rev Food Sci Nutr 57, 2404-2411.
Buenrostro, J. D., Giresi, P. G., Zaba, L. C., Chang, H. Y., and Greenleaf, W. J. (2013). Transposition of native chromatin for fast and sensitive epigenomic profiling of open chromatin, DNA-binding proteins and nucleosome position. Nat Methods 10, 1213-1218.
Butcher, M. J., Wu, C. I., Waseem, T., and Galkina, E. V. (2016). CXCR6 regulates the recruitment of pro-inflammatory IL-17A-producing T cells into atherosclerotic aortas. Int Immunol 28, 255-261.
Cheng, X., Xu, S., Zhang, C., Qin, K., Yan, J., and Shao, X. (2020). The BRCC3 regulated by Cdk5 promotes the activation of neuronal NLRP3 inflammasome in Parkinson's disease models. Biochem Biophys Res Commun 522, 647-654.
Corces, M. R., Trevino, A. E., Hamilton, E. G., Greenside, P. G., Sinnott-Armstrong, N. A., Vesuna, S., Satpathy, A. T., Rubin, A. J., Montine, K. S., Wu, B., Kathiria, A., Cho, S. W., Mumbach, M. R., Carter, A. C., Kasowski, M., Orloff, L. A., Risca, V. I., Kundaje, A., Khavari, P. A., Montine, T. J., Greenleaf, W. J., and Chang, H. Y. (2017). An improved ATAC-seq protocol reduces background and enables interrogation of frozen tissues. Nat Methods 14, 959-962.
Corrales, R. M., De Paiva, C. S., Li, D. Q., Farley, W. J., Henriksson, J. T., Bergmanson, J. P., and Pflugfelder, S. C. (2011). Entrapment of conjunctival goblet cells by desiccation-induced cornification. Invest Ophthalmol Vis Sci 52, 3492-3499.
Coursey, T. G., Bian, F., Zaheer, M., Pflugfelder, S. C., Volpe, E. A., and De Paiva, C. S. (2016). Age-related spontaneous lacrimal keratoconjunctivitis is accompanied by dysfunctional T regulatory cells. Mucosal Immunol.
Criado, G., Risco, A., Alsina-Beauchamp, D., Pdrez-Lorenzo, M. J., Escós, A., and Cuenda, A. (2014). Alternative p38 MAPKs are essential for collagen-induced arthritis. Arthritis Rheumatol 66, 1208-1217.
De Paiva, C. S., Chotikavanich, S., Pangelinan, S. B., Pitcher, J. D., 3rd, Fang, B., Zheng, X., Ma, P., Farley, W. J., Siemasko, K. F., Niederkorn, J. Y., Stern, M. E., Li, D. Q., and Pflugfelder, S. C. (2009). IL-17 disrupts corneal barrier following desiccating stress. Mucosal Immunol 2, 243-253.
De Paiva, C. S., Corrales, R. M., Villarreal, A. L., Farley, W., Li, D. Q., Stern, M. E., and Pflugfelder, S. C. (2006a). Apical corneal barrier disruption in experimental murine dry eye is abrogated by methylprednisolone and doxycycline. Invest Ophthalmol Vis Sci 47, 2847-2856.
De Paiva, C. S., Corrales, R. M., Villarreal, A. L., Farley, W. J., Li, D. Q., Stem, M. E., and Pflugfelder, S. C. (2006b). Corticosteroid and doxycycline suppress MMP-9 and inflammatory cytokine expression, MAPK activation in the corneal epithelium in experimental dry eye. Exp Eye Res 83, 526-535.
Du, X., Tabeta, K., Mann, N., Crozat, K., Mudd, S., and Beutler, B. (2005). An essential role for Rxr alpha in the development of Th2 responses. Eur J Immunol 35, 3414-3423.
Duan, J., Chung, H., Troy, E., and Kasper, D. L. (2010). Microbial colonization drives expansion of IL-1 receptor 1-expressing and IL-17-producing gamma/delta T cells. Cell Host Microbe 7, 140-150.
Fritsche, J., Stonehouse, T. J., Katz, D. R., Andreesen, R., and Kreutz, M. (2000). Expression of retinoid receptors during human monocyte differentiation in vitro. Biochem Biophys Res Commun 270, 17-22.
Gibson, B. W., Boles, N. C., Souroullas, G. P., Herron, A. J., Fraley, J. K., Schwiebert, R. S., Sharp, J. J., and Goodell, M. A. (2015). Comparison of Cesium-137 and X-ray Irradiators by Using Bone Marrow Transplant Reconstitution in C57BL/6J Mice. Comp Med 65, 165-172.
Guaiquil, V. H., Pan, Z., Karagianni, N., Fukuoka, S., Alegre, G., and Rosenblatt, M. I. (2014). VEGF-B selectively regenerates injured peripheral neurons and restores sensory and trophic functions. Proc Natl Acad Sci USA 111, 17272-17277.
Hedges, J. F., Lubick, K. J., and Jutila, M. A. (2005). Gamma delta T cells respond directly to pathogen-associated molecular patterns. J Immunol 174, 6045-6053.
Lahn, M., Kalataradi, H., Mittelstadt, P., Pflum, E., Vollmer, M., Cady, C., Mukasa, A., Vella, A. T., Ikle, D., Harbeck, R., O'brien, R., and Born, W. (1998). Early preferential stimulation of gamma delta T cells by TNF-alpha. J Immunol 160, 5221-5230.
Lambert, S., Swindell, W. R., Tsoi, L. C., Stoll, S. W., and Elder, J. T. (2017). Dual Role of Act1 in Keratinocyte Differentiation and Host Defense: TRAF3IP2 Silencing Alters Keratinocyte Differentiation and Inhibits IL-17 Responses. J Invest Dermatol 137, 1501-1511.
Li, Z., Burns, A. R., Han, L., Rumbaut, R. E., and Smith, C. W. (2011). IL-17 and VEGF are necessary for efficient corneal nerve regeneration. Am J Pathol 178, 1106-1116.
Li, Z., Burns, A. R., Rumbaut, R. E., and Smith, C. W. (2007). gamma delta T cells are necessary for platelet and neutrophil accumulation in limbal vessels and efficient epithelial repair after corneal abrasion. Am J Pathol 171, 838-845.
Liu, W., Ouyang, X., Yang, J., Liu, J., Li, Q., Gu, Y., Fukata, M., Lin, T., He, J. C., Abreu, M., Unkeless, J. C., Mayer, L., and Xiong, H. (2009). AP-1 activated by toll-like receptors regulates expression of IL-23 p19. J Biol Chem 284, 24006-24016.
Malhotra, N., Narayan, K., Cho, O. H., Sylvia, K. E., Yin, C., Melichar, H., Rashighi, M., Lefebvre, V., Harris, J. E., Berg, L. J., and Kang, J. (2013). A network of high-mobility group box transcription factors programs innate interleukin-17 production. Immunity 38, 681-693.
Marzano, A. V., Ortega-Loayza, A. G., Heath, M., Morse, D., Genovese, G., and Cugno, M. (2019). Mechanisms of Inflammation in Neutrophil-Mediated Skin Diseases. Front Immunol 10, 1059.
Mcgeachy, M. J., Cua, D. J., and Gaffen, S. L. (2019). The IL-17 Family of Cytokines in Health and Disease. Immunity 50, 892-906.
Möhn, N., Bruni, E., Schröder, A., Frömmel, S., Gueler, F., Vieten, G., Prinz, I., Kuebler, J. F., Petersen, C., and Klemann, C. (2020). Synthetic retinoid AM80 inhibits IL-17 production of gamma delta T cells and ameliorates biliary atresia in mice. Liver Int 40, 3031-3041.
Müller, A. J., Hoffmann, C., and Hardt, W. D. (2010). Caspase-1 activation via Rho GTPases: a common theme in mucosal infections? PLoS Pathog 6, e1000795.
Nien, C. J., Massei, S., Lin, G., Liu, H., Paugh, J. R., Liu, C. Y., Kao, W. W., Brown, D. J., and Jester, J. V. (2010). The development of meibomian glands in mice. Mol Vis 16, 1132-1140.
Panigrahi, T., D'souza, S., Shetty, R., Padmanabhan Nair, A., Ghosh, A., Jacob Remington Nelson, E., Ghosh, A., and Sethu, S. (2021). Genistein-Calcitriol Mitigates Hyperosmotic Stress-Induced TonEBP, CFTR Dysfunction, VDR Degradation and Inflammation in Dry Eye Disease. Clin Transl Sci 14, 288-298.
Perez, V. L., Stern, M. E., and Pflugfelder, S. C. (2020). Inflammatory basis for dry eye disease flares. Exp Eye Res 201, 108294.
Pflugfelder, S. C., and De Paiva, C. S. (2017). The Pathophysiology of Dry Eye Disease: What We Know and Future Directions for Research. Ophthalmology 124, S4-s13.
Pflugfelder, S. C., and De Paiva, C. S. (2020). Goblet cells promote tolerance induction in the conjunctiva. Mucosal Immunol 13, 717-718.
Pflugfelder, S. C., De Paiva, C. S., Moore, Q. L., Volpe, E. A., Li, D. Q., Gumus, K., Zaheer, M. L., and Corrales, R. M. (2015). Aqueous Tear Deficiency Increases Conjunctival Interferon-gamma (IFN-gamma) Expression and Goblet Cell Loss. Invest Ophthalmol Vis Sci 56, 7545-7550.
Pflugfelder, S. C., Farley, W., Luo, L., Chen, L. Z., De Paiva, C. S., Olmos, L. C., Li, D. Q., and Fini, M. E. (2005). Matrix metalloproteinase-9 knockout confers resistance to corneal epithelial barrier disruption in experimental dry eye. Am J Pathol 166, 61-71.
Pflugfelder, S. C., and Stem, M. E. (2020). The cornea in keratoconjunctivitis sicca. Exp Eye Res 201, 108295.
Powolny-Budnicka, I., Riemann, M., Tanzer, S., Schmid, R. M., Hehlgans, T., and Weih, F. (2011). RelA and RelB transcription factors in distinct thymocyte populations control lymphotoxin-dependent interleukin-17 production in γδ T cells. Immunity 34, 364-374.
Raverdeau, M., and Mills, K. H. (2014). Modulation of T cell and innate immune responses by retinoic Acid. J Immunol 192, 2953-2958.
Roszer, T., Menendez-Gutierrez, M. P., Cedenilla, M., and Ricote, M. (2013). Retinoid X receptors in macrophage biology. Trends Endocrinol Metab 24, 460-468.
Stem, M. E., Beuerman, R. W., Fox, R. I., Gao, J., Mircheff, A. K., and Pflugfelder, S. C. (1998). The pathology of dry eye: the interaction between the ocular surface and lacrimal glands. Cornea 17, 584-589.
Suryawanshi, A., Veiga-Parga, T., Reddy, P. B., Rajasagi, N. K., and Rouse, B. T. (2012). IL-17A differentially regulates corneal vascular endothelial growth factor (VEGF)-A and soluble VEGF receptor 1 expression and promotes corneal angiogenesis after herpes simplex virus infection. J Immunol 188, 3434-3446.
Tian, S., Chen, S., Feng, Y., and Li, Y. (2021). The Interactions of Small Proline-Rich Proteins with Late Cornified Envelope Proteins are Involved in the Pathogenesis of Psoriasis. Clin Cosmet Investig Dermatol 14, 1355-1365.
Wu, P., Wu, D., Ni, C., Ye, J., Chen, W., Hu, G., Wang, Z., Wang, C., Zhang, Z., Xia, W., Chen, Z., Wang, K., Zhang, T., Xu, J., Han, Y., Zhang, T., Wu, X., Wang, J., Gong, W., Zheng, S., Qiu, F., Yan, J., and Huang, J. (2014). γδT17 cells promote the accumulation and expansion of myeloid-derived suppressor cells in human colorectal cancer. Immunity 40, 785-800.
Xiao, Y., De Paiva, C. S., Yu, Z., Guimaraes De Souza, R., Li, D. Q., and Pflugfelder, S. C. (2018). Goblet cell produced retinoic acid suppresses CD86 expression and IL-12 production in bone marrow derived cells. Int Immunol 25, 457-470.
Yan, F., Powell, D. R., Curtis, D. J., and Wong, N. C. (2020). From reads to insight: a hitchhiker's guide to ATAC-seq data analysis. Genome Biol 21, 22.
Zaheer, M., Wang, C., Bian, F., Yu, Z., Hernandez, H., De Souza, R. G., Simmons, K. T., Schady, D., Swennes, A. G., Pflugfelder, S. C., Britton, R. A., and De Paiva, C. S. (2018). Protective role of commensal bacteria in Sjogren Syndrome. J Autoimmun 93, 45-56.
Zhang, X., Volpe, E. A., Gandhi, N. B., Schaumburg, C. S., Siemasko, K. F., Pangelinan, S. B., Kelly, S. D., Hayday, A. C., Li, D. Q., Stem, M. E., Niederkom, J. Y., Pflugfelder, S. C., and De Paiva, C. S. (2012). NK cells promote Th-17 mediated corneal barrier disruption in dry eye. PLoS One 7, e36822.
Zheng, X., De Paiva, C. S., Rao, K., Li, D.-Q., Farley, W. J., Stern, M., and Pflugfelder, S. C. (2010). Evaluation of the transforming growth factor-beta activity in normal and dry eye human tears by CCL-185 cell bioassay. Cornea 29, 1048-1054.
Zuberbuehler, M. K., Parker, M. E., Wheaton, J. D., Espinosa, J. R., Salzler, H. R., Park, E., and Ciofani, M. (2019). The transcription factor c-Maf is essential for the commitment of IL-17-producing Tb T cells. Nat Immunol 20, 73-85.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for treating or preventing or reducing the risk of having an eye disorder in an individual comprising administering to the individual an effective amount of a composition comprising one or more retinoid X receptor (RXR) agonists.

2. The method of claim 1, wherein the RXR agonist is an RXRα agonist.

3. The method of claim 1 or 2, wherein the RXR agonist comprises 9-cis retinoic acid, oleic acid, omega-3 docosahexaenoic acid, vitamin D, bexarotene, taxerotene, honokiol, AM80, rosiglitazone, Drupanin, garcinoic acid, NEt-3IB, or a combination thereof.

4. The method of any one of claims 1-3, wherein the therapeutic composition is administered to one or both eyes of the individual.

5. The method of claim 4, wherein the therapeutic composition is administered as an eye drop.

6. The method of claim 5, wherein the eye drop comprises a microdrop.

7. The method of any one of claims 1-4, wherein the therapeutic composition is administered as an ointment or cream.

8. The method of any one of claims 1-4, wherein the therapeutic composition is administered by subconjunctival injection.

9. The method of any one of claims 1-8, wherein the therapeutic composition comprises approximately 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 ng or μg of the RXR agonist.

10. The method of any one of claims 1-9, wherein the individual has dry eye disease, Sjogren's Syndrome, Meibomian gland disease, unstable tear film, tear dysfunction, or an ocular surface inflammatory condition, vitamin A deficiency, chemical corneal injury, thermal corneal injury, cornea inflammation following bacterial, fungal or viral infection, corneal neovascularization, or a combination thereof.

11. The method of any one of claims 1-10, wherein the composition is administered once a day.

12. The method of any one of claims 1-10, wherein the composition is administered more than once.

13. The method of claim 12, wherein the composition is administered 1, 2, 3, 4, or more times per day.

14. The method of any one of claims 1-13, wherein the composition is administered with another therapy.

15. The method of claim 14, wherein the composition is administered with a corticosteroid, cyclosporine A, a tetracycline, lifitegrast, or a combination thereof

16. The method of claim 15, wherein the tetracycline comprises minocycline and/or doxycycline.

17. The method of any one of claims 1-16, wherein the individual has a condition that predisposes the individual to the eye disorder.

18. The method of claim 17, wherein the condition comprises corneal inflammation, Sjogren syndrome, rheumatoid arthritis, systemic lupus, erythematosus, systemic sclerosis, graft versus host disease, and/or Steven's Johnson syndrome.
